(19) United States
(12) Patent Application Publication
(10) Pub. No.: US 2024/0389835 A1
(43) Pub. Date: Nov. 28, 2024

(54) Steerable instrument for endoscopic or invasive applications

(71) Applicant: Fortimedix Assets II B.V., Geleen (NL)

(72) Inventors: Mattheus Hendrik Louis Thissen, Swalmen (NL); Roy Christiaan Louisa Velter, Nuth (NL)

(73) Assignee: Fortimedix Assets II B.V., Geleen (NL)

(21) Appl. No.: 18/576,753

(22) PCT Filed: Jul. 14, 2022
(86) PCT No.: PCT/NL2022/050410

§ 371 Date: Jan. 5, 2024

(30) Foreign Application Priority Data
Jul. 15, 2021 (NL) 2028739

(51) Int. Cl. A61B 1/00; A61B 17/00

(52) U.S. Cl. CPC A61B 1/00131; A61B 2017/00305

(57) ABSTRACT

A cylindrical instrument has a tube with a movable element (1677; 16(2)) and first further element (1675; 16(1); 16(3)). The movable element (1677; 16(2)) has a movable element extending portion (1603a; 1702a1; 2002b) adjacent to a movable element indented portion (1603b; 1702b1; 2002a/2002c). In a manufactured state the movable element extending portion (1603a; 1702a1; 2002b) is located opposite a first further element indented portion (1601b; 1701b; 2001b) at a first distance and the movable element indented portion (1603b; 1702b1; 2002a/2002c) is located opposite a first further element extending portion (1601a; 1701a; 1701c; 2001a/2001c) at a second distance. Relative sideways movement between the movable element (1677; 16(2)) and the first further element (1675; 16(1); 16(3)) is possible such that when the relative sideways movement is larger than a predetermined distance the movable element extending portion (1603a; 1702a1; 2002b) is opposite the first further element extending portion (1601a; 1701a; 1701c; 2001a/2001c) at a third distance which is smaller than the first distance.

### FIELD OF THE INVENTION

The present invention relates to a steerable instrument for endoscopic and/or invasive type of applications, such as in surgery. The steerable instrument according to the invention can be used in both medical and non-medical applications. Examples of the latter include inspection and/or repair of mechanical and/or electronic hardware at locations that are difficult to reach. Hence, terms used in the following description such as endoscopic application or invasive instrument, must be interpreted in a broad manner.

### BACKGROUND ART

Transformation of surgical interventions that require large incisions for exposing a target area into minimal invasive surgical interventions, i.e. requiring only natural orifices or small incisions for establishing access to the target area, is a well-known and ongoing process. In performing minimal invasive surgical interventions, an operator such as a physician, requires an access device that is arranged for introducing and guiding invasive instruments into the human or animal body via an access port of that body. In order to reduce scar tissue formation and pain to a human or animal patient, the access port is preferably provided by a single small incision in the skin and underlying tissue. In some applications, a natural orifice of the body can be used as an entrance. Furthermore, the access device preferably enables the operator to control one or more degrees of freedom that the invasive instruments offer. In this way, the operator can perform required actions at the target area in the human or animal body in an ergonomic and accurate manner with a reduced risk of clashing of the instruments used.

Surgical invasive instruments and endoscopes are well-known in the art. Both the invasive instruments and endoscopes can comprise a steerable tube that enhances its navigation and steering capabilities. Such a steerable tube may comprise a proximal end part including at least one flexible zone, a distal end part including at least one flexible zone, and an intermediate part, wherein the steerable tube further comprises a steering arrangement that is adapted for translating a deflection of at least a part of the proximal end part relative to the intermediate part into a related deflection of at least a part of the distal end part. Alternatively, the distal flexible zone may be steered by a robotic instrument arranged at the proximal end of the steerable instrument.

Steerable invasive instruments may comprise a handle that is arranged at the proximal end part of the steerable tube for steering the tube and/or for manipulating a tool that is arranged at the distal end part of the steerable tube. Such a tool can for example be a camera, a manual manipulator, e.g. a pair of scissors, forceps, or manipulators using an energy source, e.g. an electrical, ultrasonic or optical energy source.

Furthermore, such a steerable tube may comprise a number of co-axially arranged cylindrical elements including an outer cylindrical element, an inner cylindrical element and one or more intermediate cylindrical elements depending on the number of flexible zones in the proximal and distal end parts of the tube and the desired implementation of the steering members of the steering arrangement, i.e. all steering members can be arranged in a single intermediate cylindrical element or the steering members are divided in different sets and each set of steering members is arranged, at least in part, in a different or the same intermediate cylindrical element. In most prior art devices, the steering arrangement comprises conventional steering cables with, for instance, sub 1 mm diameters as steering members, wherein the steering cables are arranged between related flexible zones at the proximal and distal end parts of the tube. Other steering units at the proximal end, like ball shaped steering units or robot driven steering units, may be applied instead.

However, as steering cables have many well-known disadvantages, for some applications one may want to avoid them and to implement the steering members by one or more sets of longitudinal steering elements that form integral parts of the one or more intermediate cylindrical elements. In some embodiments, the present invention uses this latter technology too. Each of the intermediate cylindrical elements including the longitudinal steering elements can be fabricated either by using a suitable material addition technique, such as injection molding or plating, or by starting from a tube and then using a suitable material removal technique, such as laser cutting, photochemical etching, deep pressing, conventional chipping techniques such as drilling or milling or high-pressure water jet cutting systems. Longitudinal steering elements manufactured in that way are, then, implemented as longitudinal strips resulting from the tube material, and can be used as pulling/pushing wires. Of the aforementioned material removal techniques, laser cutting is very advantageous as it allows a very accurate and clean removal of material under reasonable economic conditions.

The inner and outer cylindrical elements may be manufactured from tubes too. These tubes should be flexible at locations where the distal end, and possibly the proximal end too, of the instrument is bendable. Also at other locations where the instrument should be flexible, the inner and outer cylindrical elements should be flexible. This can be implemented by providing the inner and outer cylindrical elements with hinges at these flexible locations. Such hinges may result from (laser) cutting predetermined patterns in the tube. Many different patterns are known from the prior art. Which pattern to use depends on design requirements at the location concerned including but not limited to the required bending angle, bending flexibility, longitudinal stiffness, and radial stiffness.

Further details regarding the design and fabrication of the abovementioned steerable tube and the steering arrangement thereof have been described for example in WO 2009/112060 A1, WO 2009/127236 A1, U.S. Ser. No. 13/160,949, and U.S. Ser. No. 13/548,935 of the applicant, all of which are hereby incorporated by reference in their entirety.

In mechanical mechanisms like steerable instruments, the management of play between parts is a critical factor in obtaining optimum performance. Play has a direct influence on for example frictions, movements and positioning accuracy. When steerable instruments are made conventionally, from separate parts that will be assembled after part manufacturing, play can be managed by defining the correct dimensions of these parts and allowable tolerances. During assembly one can also adjust positions of parts with respect to each other and fix them in place to set a desired amount of play.

When one manufactures a steerable instrument as per the above mentioned patent documents with for example a laser cutting process, all parts forming the mechanism are created, in a pre-assembled state, by removing material out of a tube's wall. This will result in parts like longitudinal steering elements (strips) and hinges which are separated by an amount of play created by the material removal process and this play has a minimum width equal to or larger than the width of for example the laser cutting beam. This play can have disadvantages for the product performance. For example, when a steerable instrument is made with multiple hinges in the flexible sections, the play per hinge times the number of hinges over the length of the instrument might result in an unacceptable total play in the instrument, both in the longitudinal and tangential (circumferential) direction of the instrument.

US2014/0018620 discloses a steerable instrument comprising a coupling arrangement operable as a steering wire length compensation unit in its handle. The coupling arrangement enables the length of at least one steering wire of the set of steering wires to be adjusted. The coupling arrangement may comprise longitudinal protrusions that are interdigitatedly arranged with the steering wires. Both the protrusions and the steering wires comprise serrated portions having interlocking structures, which may have the form of triangles, for establishing a form-closed coupling upon engagement of the steering wires and the longitudinal protrusions. In a state when the steerable instrument is being inserted into a curved canal, e.g. in a human body, the steering wires and the longitudinal protrusions are not engaging one another and allow them to move freely with respect to each other. During inserting the steering wires may obtain different lengths inside its section located in the canal causing the steering wires to have different longitudinal positions in the handle. Once the steerable instrument is inserted in the desired position in the canal, the individual steering wires are locked with the coupling arrangement and the instrument is ready to be used. The locking is done by exerting a radially inwardly directed force on the coupling arrangement such that the steering wires and longitudinal protrusions are moving towards the central axis and, thus, also moving tangentially towards one another causing the serrated portions to lock into one another. During operation of the steerable instrument, i.e., while steering one or more portions of the steerable instrument with the steering wires, the individual steering wires that are locked to one another remain locked and are incapable of moving relative to one another at the locked location. I.e., only in a pre-operation state, the unlocked steering wires are capable of longitudinal movements relative to the coupling arrangement. In operation they are locked and no such mutual longitudinal movement is possible anymore at the locked locations.

### SUMMARY OF THE INVENTION

It is an object of the invention to provide a steerable instrument for endoscopic and/or invasive type of applications where at least one of the above mentioned problems are solved or at least reduced.

In particular, it is an object of the invention to provide a steerable instrument having an optimized performance with respect to at least one of obtainable distal tip payloads, tip steering accuracy and repeatability, rotational positioning accuracy and repeatability, longitudinal positioning accuracy and repeatability and durability.

To that end, independent aspects of the invention are defined in independent claims whereas dependent claims relate to advantageous embodiments.

The device with reduced play has two different states. The first state is called the manufactured state (alternatively, one may call it the “resting state”) which is the one obtained directly after manufacturing and the second state is one of reduced play in which the distance between two opposing extending portions is smaller than in the manufactured state. The reduced-play status coincides with an operation mode of the steerable instrument in which two opposing extending portions are sideways movable relative to one another (e.g. two portions of a hinge or two adjacent longitudinal elements like steering wires) along a predetermined maximum mutual displacement limit. In this reduced-play state, the two opposing extending portions slide along one another. There may be a third state between the manufactured state and the reduced-play state in which the distance between two opposing extending portions is larger than in the reduced-play state but smaller than in the manufactured state.

The movable element and the first further element may be opposite portions of a hinge and the operating of the cylindrical instrument then causes a deflection between the opposite portions of the hinge, wherein the predetermined maximum operating limit is a maximum deflection angle between the opposite portions of the hinge.

Then, the maximum deflection angle may have a value in a range of at least one of −2 to −45 degrees and +2 to +45 degrees.

The movable element may be a first longitudinal element extending in the longitudinal direction of the tube and the operating of the cylindrical instrument then causes a mutual longitudinal displacement between the longitudinal element and the first further element and the predetermined maximum operating limit is a maximum mutual longitudinal displacement.

The longitudinal element may be attached to a bendable portion of the tube at a distal end of the tube such as to transfer a longitudinal movement of the longitudinal element to a bending of the bendable portion.

Then, the maximum mutual longitudinal displacement may have a value in a range of at least one of −0.5 to −40 mm and +0.5 to +40 mm. This maximum operating limit of the movable element relative to the other element may depend on the longitudinal location in the instrument, i.e., for instance, a mutual longitudinal displacement between a steering wire and an adjacent element (e.g. another steering wire) may be much larger at the proximal end than at the distal end.

The predetermined maximum operating limit depends on the design specifications of the steerable invasive instrument, e.g., expressed in terms of maximum deflection angle of the steerable tip and maximum bending angle of adjacent hinge portions in a flexible body section of the instrument.

In this application, the terms “proximal” and “distal” are defined with respect to an operator, e.g. a robot or physician that operates the instrument or endoscope. For example, a proximal end part is to be construed as a part that is located near the robot or physician and a distal end part as a part located at a distance from the robot or physician, i.e., in the area of operation.

### BRIEF DESCRIPTION OF THE DRAWINGS

Further features and advantages of the invention will become apparent from the description of the invention by way of non-limiting and non-exclusive embodiments. These embodiments are not to be construed as limiting the scope of protection. The person skilled in the art will realize that other alternatives and equivalent embodiments of the invention can be conceived and reduced to practice without departing from the scope of the present invention. Embodiments of the invention will be described with reference to the figures of the accompanying drawings, in which like or same reference symbols denote like, same or corresponding parts, and in which:

FIG. 1 shows a schematic cross sectional view of an invasive instrument assembly having one bendable distal end portion and one proximal end portion which controls the bending of the bendable distal end portion by means of strips cut out in a cylindrical element.

FIG. 2 shows a schematic overview of three cylindrical elements from which the instrument of FIG. 1 may be manufactured.

FIG. 3a shows a portion of an intermediate cylindrical element of the instrument of FIGS. 1 and 2.

FIG. 3b shows an alternative example of an intermediate cylindrical element of such an instrument.

FIG. 4 shows an example intermediate cylindrical element and an inner cylindrical element inserted in the intermediate cylindrical element.

FIG. 5 shows an outside view of a steerable invasive instrument assemble having two steerable bendable distal end portions and two proximal flexible control portions.

FIG. 6 shows an enlarged view of the distal tip of the instrument shown in FIG. 5.

FIG. 7 shows a cross section view through the invasive instrument shown in FIG. 5.

FIGS. 8 and 9 show examples of how the invasive instrument of FIGS. 5 and 7 can bend.

FIG. 10 shows an alternative embodiment of the invasive instrument shown in FIGS. 5-9, wherein at least a portion of an intermediate section between the distal end and the proximal end is flexible too.

FIGS. 11 and 12 show schematic examples of using an invasive instrument as an endoscopic surgical instrument in which the intermediate section between the distal end and the proximal end is flexible too such that the invasive instrument can be inserted in a natural body canal like the intestinal canal, and the oesophagus.

FIGS. 13a-13c explain how cutting patterns in tubes to manufacture hinges may result in play in the instrument in use.

FIGS. 14a and 14b, respectively, show portions of the instrument as indicated with XIVa in FIG. 2 and XIVb and 1, respectively, to explain radial play in embodiments of invasive instruments.

FIGS. 15a-15f show prior art examples of how specially designed cutting patterns may counteract play in hinges in a cylindrical element.

FIGS. 16a and 16e show further examples of how specially designed cutting patterns may counteract play in hinges in a cylindrical element. FIG. 16a shows the manufactured state and FIG. 16b the reduced-play state.

FIGS. 17a-17f and 18a, 18d show examples of how specially designed cutting patterns may counteract play between longitudinal elements in a cylindrical element. FIGS. 17a, 17d, 17e, 18a, 18c show the manufactured state and FIGS. 17b, 17c, 17f, 18b, 18d the reduced-play state.

FIGS. 19a and 19b show reduction of radial play between a first cylindrical element and a second cylindrical element surrounding the first one.

FIGS. 20a-20c show an embodiment in which longitudinal steering elements have a tapered form.

FIGS. 21 and 22 show embodiments in which fracture elements are applied in the instrument.

FIGS. 23a-23e and 24a-24e show embodiments of reducing play between longitudinal steering elements.

FIGS. 25a-25d, and 26a, 26b show embodiments in which play between parts in an instrument that is made from tubes is managed by using several tubes surrounding one another to set play between parts at the desired magnitude. FIGS. 26c and 26d show a variant to the embodiment of FIGS. 26a, 26b.

### DESCRIPTION OF EMBODIMENTS

For the purpose of the present document, the terms cylindrical element and tube may be used interchangeably, i.e., like the term tube a cylindrical element also refers to a physical entity. The invention will be explained with reference to longitudinal steering elements which are cut from such cylindrical elements and are operative as push and/or pull wires to transfer longitudinal movement of the steering elements at the proximal end of the instrument to the distal end to thereby control bending of one or more flexible distal end portions. Embodiments in which reduction of play in hinges is explained can also be implemented with wires made in a classic way and not resulting from cutting them out of a tube.

### Instruments in which the Invention can be Applied

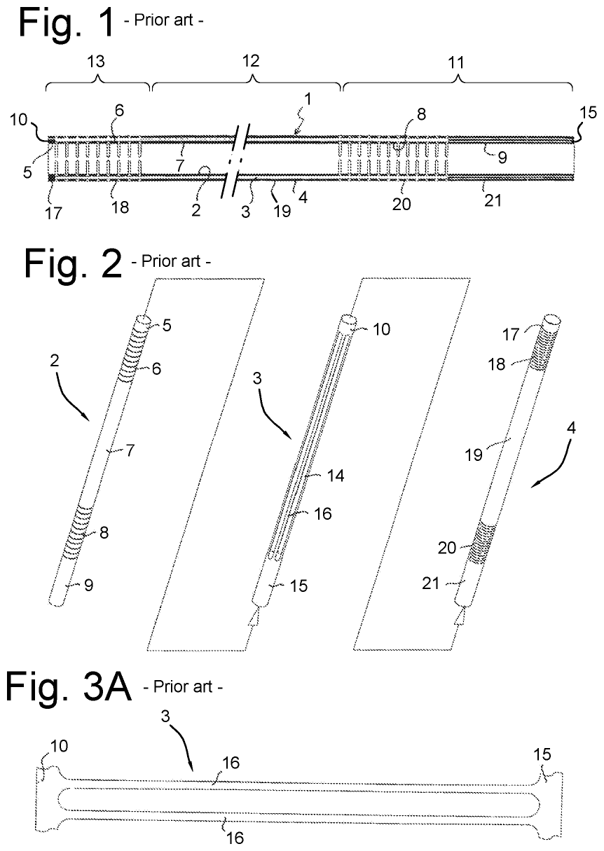

FIGS. 1, 2, 3a, and 3b are known from WO2009/112060. They are explained in detail because the present invention can be applied in this type of instruments.

FIG. 1 shows a longitudinal cross-section of a prior art steerable instrument comprising three co-axially arranged cylindrical elements, i.e. inner cylindrical element 2, intermediate cylindrical element 3 and outer cylindrical element 4. Suitable materials to be used for making the cylindrical elements 2, 3, and 4 include stainless steel, cobalt-chromium, shape memory alloy such as Nitinol®, plastic, polymer, composites or other materials that can be shaped by material removal processes like laser cutting or EDM. Alternatively, the cylindrical elements can be made by a 3D printing process or other known material deposition processes.

The inner cylindrical element 2 comprises a first rigid end part 5, which is located at a distal end part 13 of the instrument, a first flexible part 6, an intermediate rigid part 7 located at an intermediate part 12 of the instrument, a second flexible part 8 and a second rigid end part 9, which is located at a proximal end part 11 of the instrument.

The outer cylindrical element 4 also comprises a first rigid end part 17, a first flexible part 18, an intermediate rigid part 19, a second flexible part 20 and a second rigid end part 21. The lengths of the parts 5, 6, 7, 8, and 9, respectively, of the cylindrical element 2 and the parts 17, 18, 19, 20, and 21, respectively, of the cylindrical element 4 are, preferably, substantially the same so that when the inner cylindrical element 2 is inserted into the outer cylindrical element 4, these different respective parts are longitudinally aligned with each other.

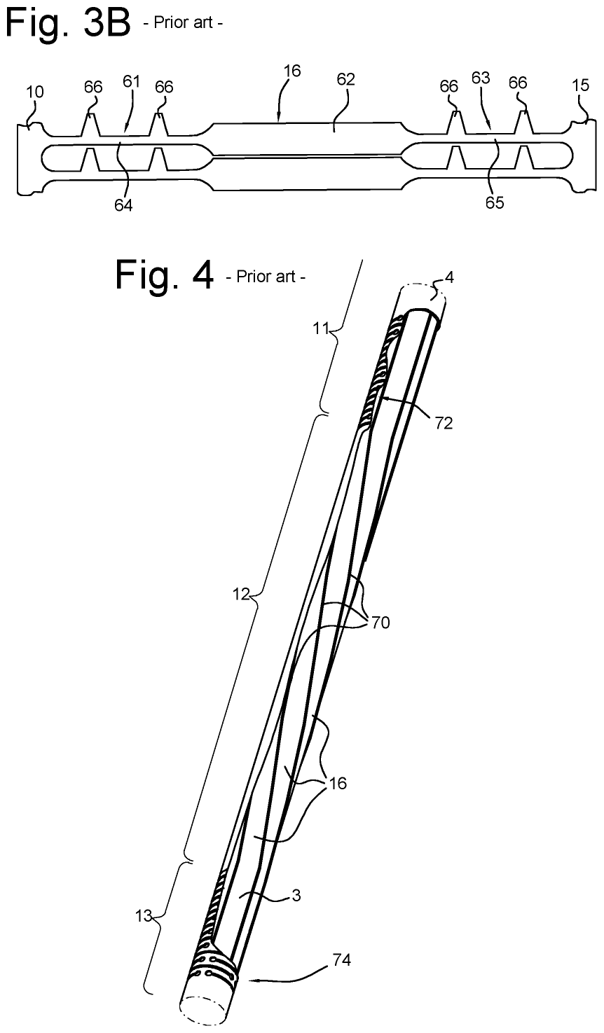

The intermediate cylindrical element 3 also has a first rigid end part 10 and a second rigid end part 15 which in the assembled condition are located between the corresponding rigid parts 5, 17 and 9, 21 respectively of the two other cylindrical elements 2, 4. The intermediate part 14 of the intermediate cylindrical element 3 comprises one or more separate longitudinal steering elements 16 which can have different forms and shapes as will be explained below. In FIG. 3a, three such longitudinal steering elements 16 are shown. After assembly of the three cylindrical elements 2, 3 and 4 whereby the element 2 is inserted in the element 3 and the two combined elements 2, 3 are inserted into the element 4, at least the first rigid end part 5 of the inner cylindrical element 2, the first rigid end part 10 of the intermediate cylindrical element 3 and the first rigid end part 17 of the outer cylindrical element 4 at the distal end of the instrument are attached to each other, e.g., by means of glue or one or more laser welding spots. In the embodiment shown in FIGS. 1 and 2, also the second rigid end part 9 of the inner cylindrical element 2, the second rigid end part 15 of the intermediate cylindrical element 3 and the second rigid end part 21 of the outer cylindrical element 4 at the proximal end of the instrument are attached to each other, e.g. by means of glue or one or more laser welding spots, such that the three cylindrical elements 2, 3, 4 form one integral unit.

In the embodiment shown in FIG. 2 the intermediate part 14 of intermediate cylindrical element 3 comprises a number of longitudinal steering elements 16 with a uniform cross-section so that the intermediate part 14 has the general shape and form as shown in the unrolled condition of the intermediate cylindrical element 3 in FIG. 3a. From FIG. 3a it also becomes clear that the intermediate part 14 is formed by a number of over the circumference of the intermediate cylindrical part 3, possibly equally, spaced parallel longitudinal steering elements 16. Advantageously, the number of longitudinal steering elements 16 is at least three, so that the instrument becomes fully controllable in any direction, but any higher number is possible as well. The number of longitudinal steering elements 16 may, e.g., be six or eight.

It is observed that the longitudinal steering elements 16 need not have a uniform cross section across their entire length. They may have a varying width along their length, possibly such that at one or more locations adjacent longitudinal steering elements 16 are only separated by a small slot resulting from the laser cutting in the cylindrical element 3. These wider portions of the longitudinal steering elements, then, operate as spacers to prevent adjacent longitudinal steering elements 16 from buckling in a tangential direction in a pushed state. Spacers may, alternatively, be implemented in other ways.

An embodiment with spacers is shown in FIG. 3b which shows two adjacent longitudinal steering elements 16 in an unrolled condition. In the embodiment shown in FIG. 3b each longitudinal steering element 16 is composed of three portions 61, 62 and 63, co-existing with the first flexible part 6, 18 the intermediate rigid part 7, 19 and the second flexible part 8, 20 respectively. In the portion 62 coinciding with the intermediate rigid portion, each pair of adjacent longitudinal steering elements 16 is almost touching each other in the tangential direction so that in fact only a narrow slot is present there between just sufficient to allow independent movement of each longitudinal steering element. The slot results from the manufacturing process and its width is, e.g., caused by the diameter of a laser beam cutting the slot.

In the other two portions 61 and 63 each longitudinal steering element consists of a relatively small and flexible part 64, 65 as seen in circumferential direction, so that there is a substantial gap between each pair of adjacent flexible parts, and each flexible part 64, 65 is provided with a number of spacers 66, extending in the tangential direction and almost bridging completely the gap to the adjacent flexible part 64, 65. Because of these spacers 66 the tendency of the longitudinal steering elements 16 in the flexible portions of the instrument to shift in tangential direction is suppressed and tangential direction control is improved. The exact shape of these spacers 66 is not very critical, provided they do not compromise flexibility of flexible parts 64 and 65. The spacers 66 may form an integral part with the flexible parts 64, 65 and may result from a suitable laser cutting process too.

In the embodiment shown in FIG. 3b the spacers 66 are extending towards one tangential direction as seen from the flexible part 64, 65 to which they are attached. It is however also possible to have these spacers 66 extending to both circumferential directions starting from one flexible part 64, 65. By using this it is either possible to have alternating types of flexible parts 64, 65 as seen along the tangential direction, wherein a first type is provided at both sides with spacers 66 extending until the next flexible part, and a second intermediate set of flexible parts 64, 65 without spacers 66. Otherwise it is possible to have flexible parts with cams at both sides, where as seen along the longitudinal direction of the instrument the cams originating from one flexible part are alternating with spacers originating from the adjacent flexible parts. It is obvious that numerous alternatives are available.

The production of such an intermediate part is most conveniently done by injection moulding or plating techniques or starting from a cylindrical tube with the desired inner and outer diameters and removing parts of the wall of the cylindrical tube required e.g. by laser or water cutting to end up with the desired shape of the intermediate cylindrical element 3. However, alternatively, any 3D printing method can be used.

The removal of material can be done by means of different techniques such as laser cutting, photochemical etching, deep pressing, conventional chipping techniques such as drilling or milling, high pressure water jet cutting systems or any suitable material removing process available. Preferably, laser cutting is used as this allows for a very accurate and clean removal of material under reasonable economic conditions. The above mentioned processes are convenient ways as the cylindrical element 3 can be made so to say in one process, without requiring additional steps for connecting the different parts of the intermediate cylindrical element as required in the conventional instruments, where conventional steering cables must be connected in some way to the end parts. The same type of technology can be used for producing the inner and outer cylindrical elements 2 and 4 with their respective flexible parts 6, 8, 18 and 20. These flexible parts 6, 8, 18 and 20 can be manufactured as hinges resulting from cutting out any desired pattern from the cylindrical elements, e.g., by using any of the methods described in European patent application 08 004 373.0 filed on 10 Mar. 2008, page 5, lines 15-26, but any other suitable process can be used to make flexible portions.

It is observed that the instruments shown in FIGS. 4-10 are known from prior art WO2020/214027. Also in these instruments the present invention can be applied.

FIG. 4 shows an exemplary embodiment of longitudinal (steering) elements 16 that have been obtained after providing longitudinal slots 70 to the wall of the intermediate cylindrical element 3 that interconnects proximal flexible zone 14 and distal flexible zone 16 as described above. Here, longitudinal steering elements 16 are, at least in part, spiraling about a longitudinal axis of the instrument such that an end portion of a respective steering element 16 at the proximal portion of the instrument is arranged at another angular orientation about the longitudinal axis than an end portion of the same longitudinal steering element 16 at the distal portion of the instrument. Were the longitudinal steering elements 16 arranged in a linear orientation, than a bending of the instrument at the proximal portion in a certain plane would result in a bending of the instrument at the distal portion in the same plane but in a 180 degrees opposite direction. This spiral construction of the longitudinal steering elements 16 allows for the effect that bending of the instrument at the proximal portion in a certain plane may result in a bending of the instrument at the distal portion in another plane, or in the same plane in the same direction. A preferred spiral construction may be such that the end portion of a respective steering element 16 at the proximal portion of the instrument is arranged at an angularly shifted orientation of 180 degrees about the longitudinal axis relative to the end portion of the same longitudinal steering element 16 at the distal portion of the instrument. However, e.g. any other angularly shifted orientation, e.g. 90 degrees, is within the scope of this document. The slots 70 are dimensioned such that movement of a longitudinal steering element is guided by adjacent longitudinal steering elements when provided in place in a steerable instrument. However, especially at the flexible zones 13, 14 of the instrument, the width of longitudinal steering elements 16 may be less to provide the instrument with the required flexibility/bendability at those locations.

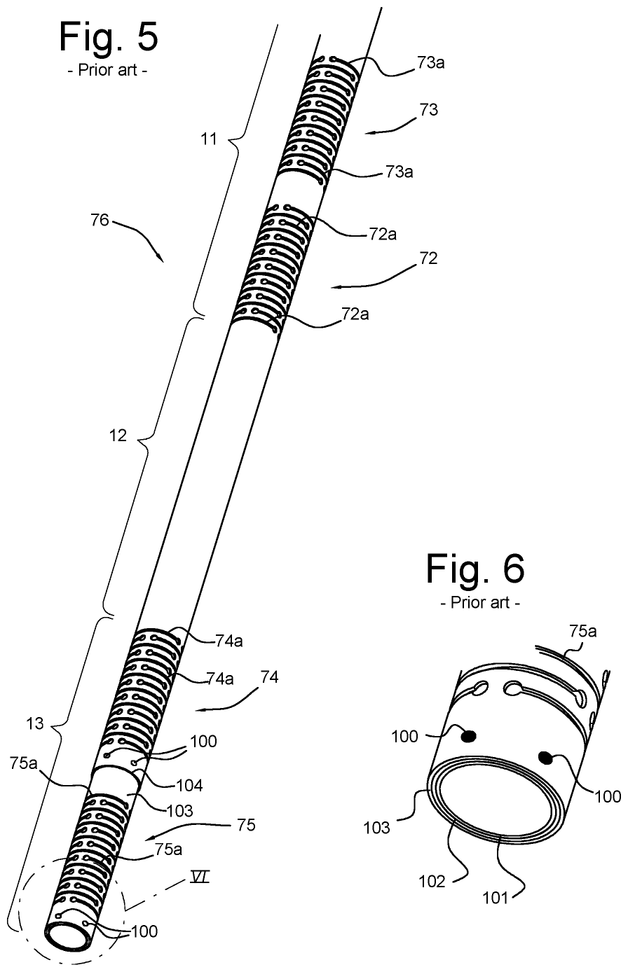

FIG. 5 provides a detailed perspective view of the distal portion of an embodiment of an elongated tubular body 76 of a steerable instrument which has two steerable distal bendable zones 74, 75 which are operated by two bendable proximal zones 72, 73, respectively. FIG. 5 shows that the elongated tubular body 76 comprises a number of co-axially arranged layers or cylindrical elements including an outer cylindrical element 104 that ends after a first distal flexible zone 74 at the distal end portion 13. The distal end portion 13 of the outer cylindrical element 104 is fixedly attached to a cylindrical element 103 located inside of and adjacent to the outer cylindrical element 104, e.g. by means of spot welding at welding spots 100. However, any other suitable attachment method can be used, including any mechanical snap fit connection or gluing by a suitable glue.

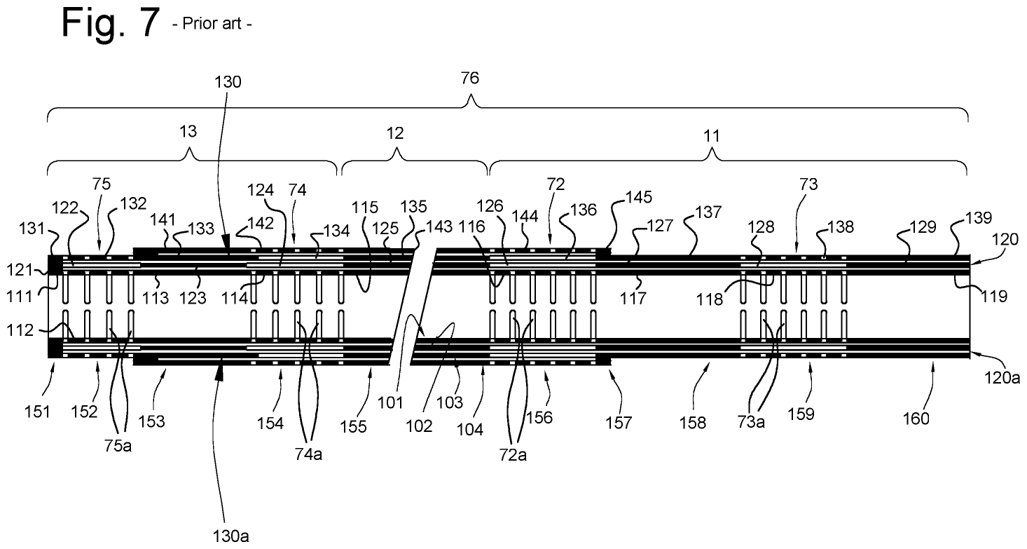

FIG. 6 provides a more detailed view of the distal end part 13 and shows that, in this embodiment, it includes three co-axially arranged layers or cylindrical elements, i.e., an inner cylindrical element 101, a first intermediate cylindrical element 102 and a second intermediate cylindrical element 103. The distal ends of inner cylindrical element 101, first intermediate cylindrical element 102 and second intermediate cylindrical element 103 are all three fixedly attached to one another. This may be done by means of spot welding at welding spots 100. However, any other suitable attachment method can be used, including any mechanical snap fit connection or gluing by a suitable glue. The points of attachment may be at the end edges of inner cylindrical element 101, first intermediate cylindrical element 102 and second intermediate cylindrical element 103, as shown in the figures. However, these points of attachment may also be located some distance away from these edges, be it, preferably, between the end edges and the locations of the flexible zone 75.

It will be clear to the skilled person that the elongated tubular body 76 as shown in FIG. 5 comprises four cylindrical elements in total. The elongated tubular body 76 according to the embodiment shown in FIG. 5 comprises two intermediate cylindrical elements 102 and 103 in which the steering members of the steering arrangement are arranged. However, extra or less cylindrical elements may be provided if desired.

The steering arrangement in the exemplary embodiment of the elongated tubular body 76 as shown in FIG. 5 comprises the two flexible zones 72, 73 at the proximal end part 11 of the elongated tubular body 76, the two flexible zones 74, 75 at the distal end part 13 of the elongated tubular body 76 and the steering members that are arranged between related flexible zones at the proximal 11 and distal 13 end parts. An exemplary actual arrangement of the steering members is shown in FIG. 7, which provides a schematic longitudinal cross-sectional view of the exemplary embodiment of the elongated tubular body 76 as shown in FIG. 5.

Flexible zones 72, 73, 74, and 75 are, in this embodiment, implemented by providing the respective cylindrical elements with slits 72a, 73a, 74a, and 75a, respectively. Such slits 72a, 73a, 74a, and 75a may be arranged in any suitable pattern such that the flexible zones 72, 73, 74, and 75 have a desired flexibility in the longitudinal and tangential direction in accordance with a desired design.

FIG. 7 shows a longitudinal cross section of the four layers or cylindrical elements mentioned above, i.e. the inner cylindrical element 101, the first intermediate cylindrical element 102, the second intermediate cylindrical element 103, and the outer cylindrical element 104.

The inner cylindrical element 101, as seen along its length from the distal end to the proximal end of the instrument, comprises a rigid ring 111, which is arranged at the distal end part 13 of the steerable instrument 10, a first flexible portion 112, a first intermediate rigid portion 113, a second flexible portion 114, a second intermediate rigid portion 115, a third flexible portion 116, a third intermediate rigid portion 117, a fourth flexible portion 118, and a rigid end portion 119, which is arranged at the proximal end portion 11 of the steerable instrument.

The first intermediate cylindrical element 102, as seen along its length from the distal end to the proximal end of the instrument, comprises a rigid ring 121, a first flexible portion 122, a first intermediate rigid portion 123, a second flexible portion 124, a second intermediate rigid portion 125, a third flexible portion 126, a third intermediate rigid portion 127, a fourth flexible portion 128, and a rigid end portion 129. The portions 122, 123, 124, 125, 126, 127 and 128 together form a longitudinal steering element 120 that can be moved in the longitudinal direction like a wire. The longitudinal dimensions of the rigid ring 121, the first flexible portion 122, the first intermediate rigid portion 123, the second flexible portion 124, the second intermediate rigid portion 125, the third flexible portion 126, the third intermediate rigid portion 127, the fourth flexible portion 128, and the rigid end portion 129 of the first intermediate element 102, respectively, are aligned with, and preferably approximately equal to the longitudinal dimensions of the rigid ring 111, the first flexible portion 112, the first intermediate rigid portion 113, the second flexible portion 114, the second intermediate rigid portion 115, the third flexible portion 116, the third intermediate rigid portion 117, the fourth flexible portion 118, and the rigid end portion 119 of the inner cylindrical element 101, respectively, and are coinciding with these portions as well. In this description “approximately equal” means that respective same dimensions are equal within a margin of less than 10%, preferably less than 5%.

Similarly, the first intermediate cylindrical element 102 comprises one or more other longitudinal steering elements of which one is shown with reference number 120a.

The second intermediate cylindrical element 103, as seen along its length from the distal end to the proximal end of the instrument, comprises a first rigid ring 131, a first flexible portion 132, a second rigid ring 133, a second flexible portion 134, a first intermediate rigid portion 135, a first intermediate flexible portion 136, a second intermediate rigid portion 137, a second intermediate flexible portion 138, and a rigid end portion 139. The portions 133, 134, 135 and 136 together form a longitudinal steering element 130 that can be moved in the longitudinal direction like a wire. The longitudinal dimensions of the first rigid ring 131, the first flexible portion 132 together with the second rigid ring 133 and the second flexible portion 134, the first intermediate rigid portion 135, the first intermediate flexible portion 136, the second intermediate rigid portion 137, the second intermediate flexible portion 138, and the rigid end portion 139 of the second intermediate cylinder 103, respectively, are aligned with, and preferably approximately equal to the longitudinal dimensions of the rigid ring 111, the first flexible portion 112, the first intermediate rigid portion 113, the second flexible portion 114, the second intermediate rigid portion 115, the third flexible portion 116, the third intermediate rigid portion 117, the fourth flexible portion 118, and the rigid end portion 119 of the first intermediate element 102, respectively, and are coinciding with these portions as well.

Similarly, the second intermediate cylindrical element 103 comprises one or more other longitudinal steering elements of which one is shown with reference number 130a.

The outer cylindrical element 104, as seen along its length from the distal end to the proximal end of the instrument, comprises a first rigid ring 141, a first flexible portion 142, a first intermediate rigid portion 143, a second flexible portion 144, and a second rigid ring 145. The longitudinal dimensions of the first flexible portion 142, the first intermediate rigid portion 143 and the second flexible portion 144 of the outer cylindrical element 104, respectively, are aligned with, and preferably approximately equal to the longitudinal dimension of the second flexible portion 134, the first intermediate rigid portion 135 and the first intermediate flexible portion 136 of the second intermediate element 103, respectively, and are coinciding with these portions as well. The rigid ring 141 has approximately the same length as the rigid ring 133 and is fixedly attached thereto, e.g. by spot welding or gluing. Preferably, the rigid ring 145 overlaps with the second intermediate rigid portion 137 only over a length that is required to make an adequate fixed attachment between the rigid ring 145 and the second intermediate rigid portion 137, respectively, e.g. by spot welding or gluing. The rigid rings 111, 121 and 131 are attached to each other, e.g., by spot welding or gluing. This may be done at the end edges thereof but also at a distance of these end edges.

In an embodiment, the same may apply to the rigid end portions 119, 129 and 139, which can be attached to one another as well in a comparable manner. However, the construction may be such that the diameter of the cylindrical elements at the proximal portion is larger, or smaller, with respect to the diameter at the distal portion. In such embodiment the construction at the proximal portion differs from the one shown in FIG. 7. As a result of the increase or decrease in diameter an amplification or attenuation is achieved, i.e., the bending angle of a flexible zone at the distal portion will be larger or smaller than the bending angle of a corresponding flexible portion at the proximal portion.

The inner and outer diameters of the cylindrical elements 101, 102, 103, and 104 are chosen in such a way at a same location along the elongated tubular body 76 that the outer diameter of inner cylindrical element 101 is slightly less than the inner diameter of the first intermediate cylindrical element 102, the outer diameter of the first intermediate cylindrical element 102 is slightly less than the inner diameter of the second intermediate cylindrical element 103 and the outer diameter of the second intermediate cylindrical element 103 is slightly less than the inner diameter of the outer cylindrical element 104, in such a way that a sliding movement of the adjacent cylindrical elements with respect to each other is possible. The dimensioning should be such that a sliding fit is provided between adjacent elements. A clearance between adjacent elements may generally be in the order of 0.02 to 0.1 mm, but depends on the specific application and material used. The clearance may be smaller than a wall thickness of the longitudinal steering elements to prevent an overlapping configuration thereof. Restricting the clearance to about 30% to 40% of the wall thickness of the longitudinal steering elements is generally sufficient.

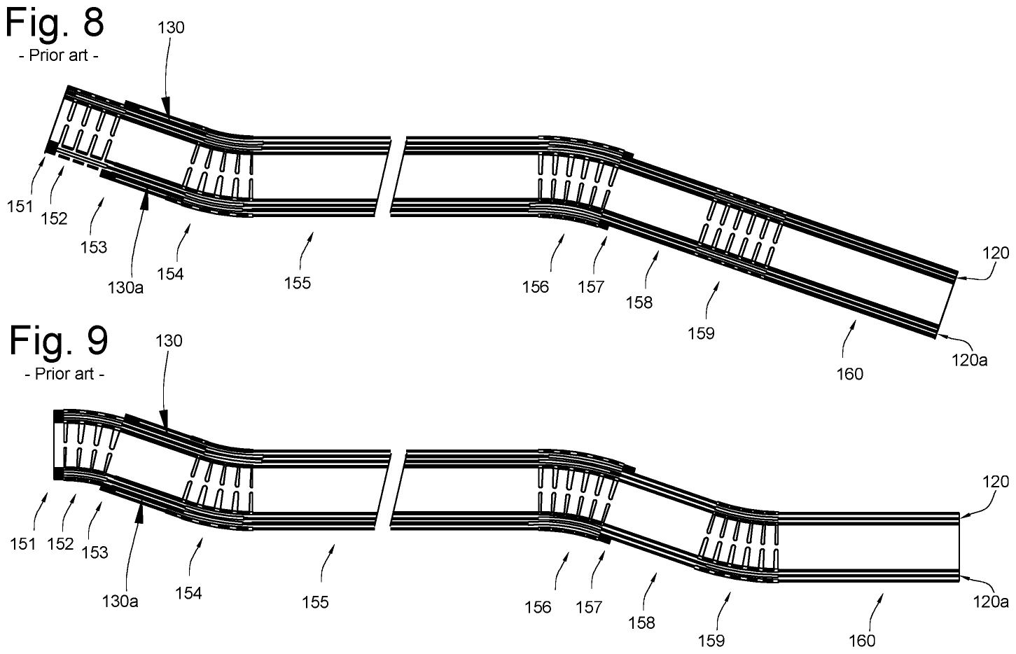

As can be seen in FIG. 7, flexible zone 72 of the proximal end part 11 is connected to the flexible zone 74 of the distal end part 13 by portions 134, 135 and 136, of the second intermediate cylindrical element 103, which form a first set of longitudinal steering elements of the steering arrangement of the steerable instrument. Furthermore, flexible zone 73 of the proximal end part 11 is connected to the flexible zone 75 of the distal end part 13 by portions 122, 123, 124, 125, 126, 127, and 128 of the first intermediate cylindrical element 102, which form a second set of longitudinal steering elements of the steering arrangement. The use of the construction as described above allows the steerable instrument 10 to be used for double bending. The working principle of this construction will be explained with respect to the examples shown in FIGS. 8 and 9.

For the sake of convenience, as shown in FIGS. 7, 8 and 9, the different portions of the cylindrical elements 101, 102, 103, and 104 have been grouped into zones 151-160 that are defined as follows. Zone 151 comprises the rigid rings 111, 121, and 131. Zone 152 comprises the portions 112, 122, and 132. Zone 153 comprises the rigid rings 133 and 141 and the portions 113 and 123. Zone 154 comprises the portions 114, 124, 134 and 142. Zone 155 comprises the portions 115, 125, 135 and 143. Zone 156 comprises the portions 116, 126, 136 and 144. Zone 157 comprises the rigid ring 145 and the parts of the portions 117, 127, and 137 coinciding therewith. Zone 158 comprises the parts of the portions 117, 127, and 137 outside zone 157. Zone 159 comprises the portions 118, 128 and 138. Finally, zone 160 comprises the rigid end portions 119, 129 and 139.

In order to deflect at least a part of the distal end part 13 of the steerable instrument, it is possible to apply a bending force, in any radial direction, to zone 158. According to the examples shown in FIGS. 8 and 9, zone 158 is bent downwards with respect to zone 155. Consequently, zone 156 is bent downwards. Because of the first set of longitudinal steering elements comprising portions 134, 135, and 136 of the second intermediate cylindrical element 103 that are arranged between the second intermediate rigid portion 137 and the second rigid ring 133, the downward bending of zone 156 is transferred by a longitudinal displacement of the first set of longitudinal steering elements into an upward bending of zone 154 with respect to zone 155. This is shown in both FIGS. 8 and 9.

It is to be noted that the exemplary downward bending of zone 156, only results in the upward bending of zone 154 at the distal end of the instrument as shown in FIG. 8. Bending of zone 152 as a result of the bending of zone 156 is prevented by zone 153 that is arranged between zones 152 and 154. When subsequently a bending force, in any radial direction, is applied to the zone 160, zone 159 is also bent. As shown in FIG. 9, zone 160 is bent in an upward direction with respect to its position shown in FIG. 8. Consequently, zone 159 is bent in an upward direction. Because of the second set of longitudinal steering elements comprising portions 122, 123, 124, 125, 126, 127 and 128 of the first intermediate cylindrical element 102 that are arranged between the rigid ring 121 and the rigid end portion 129, the upward bending of zone 159 is transferred by a longitudinal displacement of the second set of longitudinal steering elements into a downward bending of zone 152 with respect to its position shown in FIG. 8.

FIG. 9 further shows that the initial bending of the instrument in zone 154 as shown in FIG. 8 will be maintained because this bending is only governed by the bending of zone 156, whereas the bending of zone 152 is only governed by the bending of zone 159 as described above. Due to the fact that zones 152 and 154 are bendable independently with respect to each other, it is possible to give the distal end part 13 of the steerable instrument a position and longitudinal axis direction that are independent from each other. In particular the distal end part 13 can assume an advantageous S-like shape. The skilled person will appreciate that the capability to independently bend zones 152 and 154 with respect to each other, significantly enhances the maneuverability of the distal end part 13 and therefore of the steerable instrument as a whole.

Obviously, it is possible to vary the lengths of the flexible portions shown in FIGS. 7 to 9 as to accommodate specific requirements with regard to bending radii and total lengths of the distal end part 13 and the proximal end part 11 of the steerable instrument or to accommodate amplification or attenuation ratios between bending of at least a part of the proximal end part 11 and at least a part of the distal end part 13.

In the shown embodiment, the longitudinal steering elements comprise one or more sets of longitudinal steering elements that form integral parts of the one or more intermediate cylindrical elements 102, 103. Preferably, the longitudinal steering elements comprise remaining parts of the wall of an intermediate cylindrical element 102, 103 after the wall of the intermediate cylindrical element 102, 103 has been provided with longitudinal slits that define the remaining longitudinal steering elements.

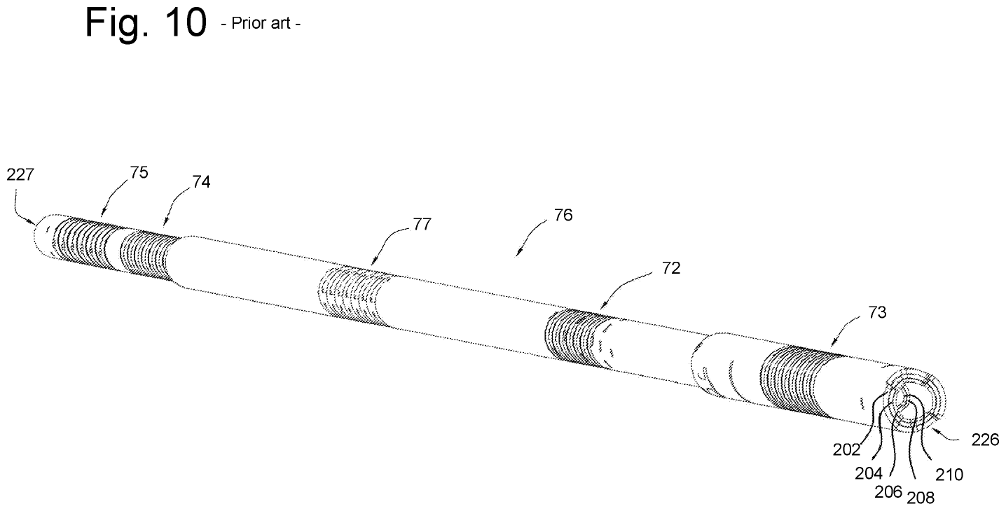

FIG. 10 shows a 3D view of an example of a steerable instrument. Like reference numbers refer to the same elements as in other figures. Their explanation is not repeated here. The instruments comprises five coaxial cylindrical elements 202-210. An inner cylindrical element 210 is surrounded by intermediate cylindrical element 208 which is surrounded by intermediate cylindrical element 206 which is surrounded by intermediate cylindrical element 204 which is, finally surrounded by outer cylindrical element 202. Inner intermediate cylindrical element may be made of a flexible spiraling spring. The proximal and distal ends, respectively, of the instrument are indicated with reference numbers 226 and 227, respectively.

As shown, here, instrument 76 comprises a flexible zone 77 in its intermediate part between flexible zone 72 and flexible zone 74. I.e., intermediate cylindrical element 204 (which is located at the outer side in the area of flexible zone 77) is provided with a slotted structure to provide intermediate cylindrical element with a desired flexibility. The longitudinal length of the slotted structure in flexible zone 77 depends on the desired application. It may be as long as the entire part between flexible zones 72 and 74. All other cylindrical elements 206, 208, 210 inside intermediate cylindrical element 204 are also flexible in flexible zone 77. Those cylindrical elements that have longitudinal steering elements in the flexible zone 77 are flexible by way of definition. Others are provided with suitable hinges, preferably made by suitable slotted structures.

Some locations to be operated in a body need specifically designed instruments. E.g., by making the intermediate part 12 of the instrument completely flexible, the instrument can also be used in areas in the body which are only accessible via curved natural access guides/channels, like the colon, the stomach via the oesophagus or the heart via curved blood vessels.

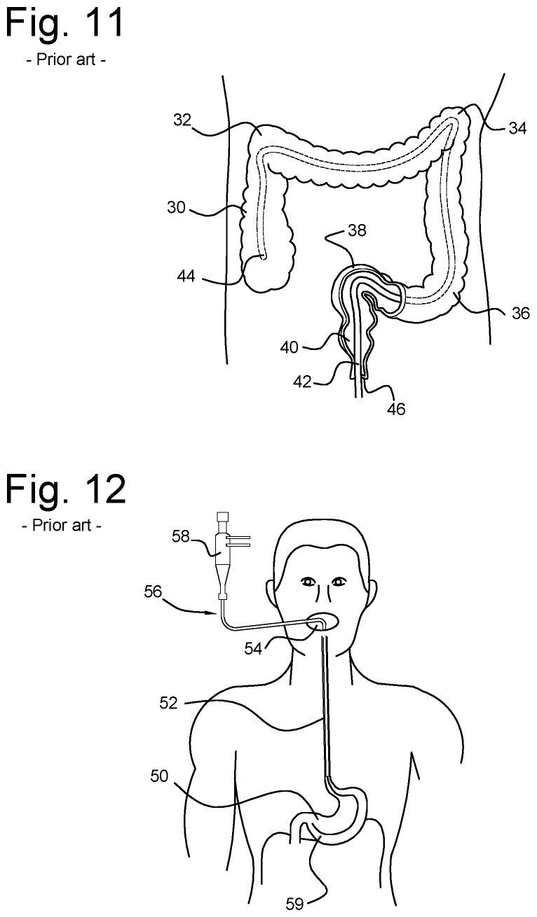

The instrument can e.g. be designed to be used as a colonoscope. FIG. 11 shows a schematic view of a colonoscope 42 in use. The colonoscope 42 is inserted into a colon 30 of a human body. Typically, the colon 30 has several almost square angled sections 32, 34, 36, and 38. If a surgeon needs to operate an area of the colon 30 upstream from square angled section 32 the colonoscope 42 needs to be inserted into the colon 30 along a distance of up to 1.5 meter. Moreover, the colonoscope 42 needs to be so flexible that it can be guided from an anus through all squared angled sections 32-38 of the colon 30 easily without risks of damaging the inner wall of the colon 30.

In operation, usually, several invasive instruments are inserted through the colonoscope 42 to provide one or more tools for some function at its distal end 44. In colonoscopy, such a tool typically includes a camera lens and a lighting element. To assist the surgeon in steering the camera view to the desired location and view in colon 30, typically, the distal end is deflectable from a longitudinal axis in all angular directions. This also holds for the inserted instruments with tools 2. That can be implemented by providing such an instruments with one or more deflectable zones, like the deflectable zones 16, 17 of the instrument shown in FIGS. 5-10. These distal deflectable zones are controlled by suitable steering cables accommodated in the instruments connected to a suitable steering mechanism at the proximal ends of the instruments.

FIG. 12 shows a schematic view of a gastroscope 56 in use. The gastroscope 56 is inserted into a stomach 50 of a human body via mouth, oral cavity/throat 54 and oesophagus 52. Especially when a surgeon needs to operate a lower portion of the stomach 50, the gastronoscope 56 needs to be guided through several curved/angled sections. Therefore, the gastroscope 56 needs to be flexible such that there is little risk of damaging inner walls of the mouth/throat 54, oesophagus 52 and stomach 50.

In operation, usually, several invasive instruments are inserted through the gastroscope 56 to provide one or more tools for some function at its distal end 59. In gastroscopy, such a tool typically includes a camera lens and a lighting element. To assist the surgeon in steering the camera view to the desired location and direction in stomach 50, typically, the distal end 59 of the gastrocope 56 is deflectable from a longitudinal axis in all angular directions. This also holds for the inserted instruments with tools 2. That can be implemented by providing such an instrument with one or more deflectable zones, like the deflectable zones 16, 17 of the instrument shown in FIGS. 5-10. These distal deflectable zones are controlled by suitable steering cables accommodated in the instruments connected to a suitable steering mechanism of these instruments.

Instruments according to the invention can be used in such colonoscopes and gastroscopes. Requirements to such an instrument may be that they show a high rotational stiffness, high longitudinal stiffness, flexibility along its entirely length and deflectability at its deflectable zones even in cases of long instruments, e.g., longer than 1 m, and with a relatively small diameter that fits to the working channels within or attached to colonoscopes and gastroscopes.

### Play in Invasive Instruments

Especially for instruments designed for applications as shown in FIG. 11, play should be kept at a minimum, both in the longitudinal and tangential direction. The less the play the more direct the control of the movement of a tool at the distal end of the instrument from the proximal end will be. The present document explains how play in such instruments results, amongst others, from slits between adjacent parts in the cylindrical elements, for instance between adjacent parts of hinges cut in a cylindrical element or longitudinal steering elements cut in a cylindrical element.

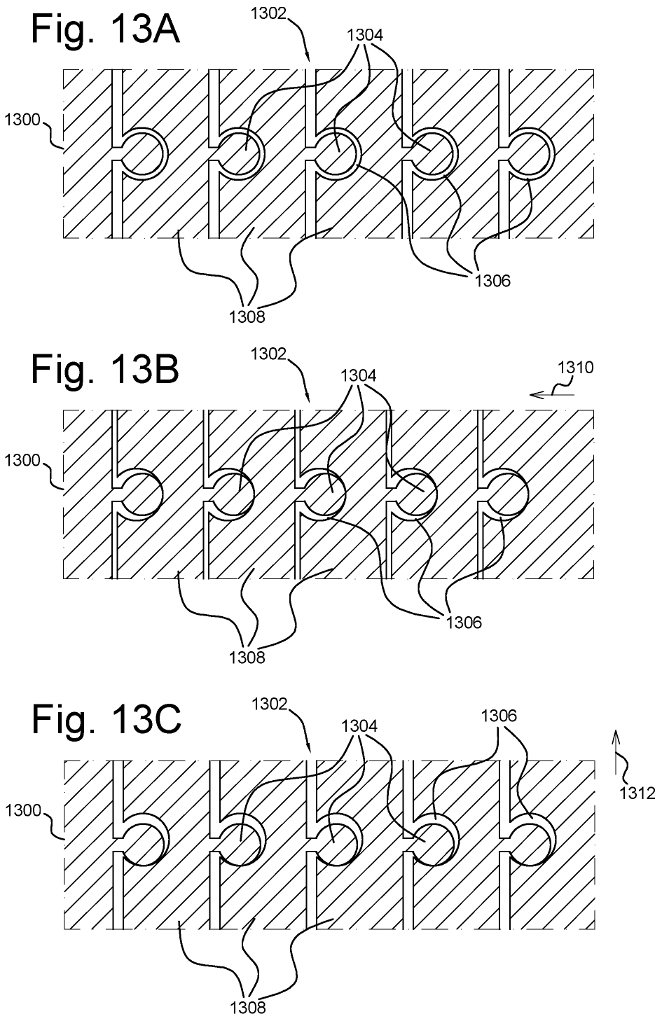

The problem of play in the longitudinal and/or tangential direction in hinges in an invasive instrument will be explained with reference to FIGS. 13a-13c. However, similar or identical problems may exist in hinges of other structures known from the prior art and/or still to be developed. The solution of play in hinges is, therefore not restricted to the example of FIGS. 13a-13c but relates to any type of hinge made in a cylindrical element (or tube).

FIGS. 13a-13c show a schematic outside view of a portion of a series of adjacent hinge segment 1308 of a hinge 1302 in a cylindrical element 1300 which can be any of the above shown cylindrical elements 2, 4, 101, 102, 103, 104, 202, 204, 206, 208, and 210. In the shown example, one of the adjacent hinge segments 1308 has a convex (circularly shaped) portion 1304 whereas the other one has a concave portion 1306 accommodating one convex portion 1304. A structure with one such convex portion 1304 inside such a concave portion 1306 can be part of a typical hinge. The convex portion 1304 can rotate to a certain extent inside the adjacent concave portion 1306 depending on how the different portions 1304 and 1306 are designed. The total bending angle the hinge 1302 can make depends on the number of hinge segments 1308.

The structure shown in FIGS. 13a-13c is, in this embodiment, present on one side of the cylindrical element 1300, and is identically present at the opposite side 180° rotated in the tangential direction in relation to FIGS. 13a-13c. In the shown embodiment, subsequent couples of one convex portion 1304 and one concave portion 1306 have the same tangential locations such that the shown hinge 1302 can only bend in the surface of the drawing. By alternating tangential locations of subsequent couples of one convex portion 1304 and one concave portion 1306 about 90°, the hinge 1302 would be bendable in all directions, as is known to a person skilled in the art.

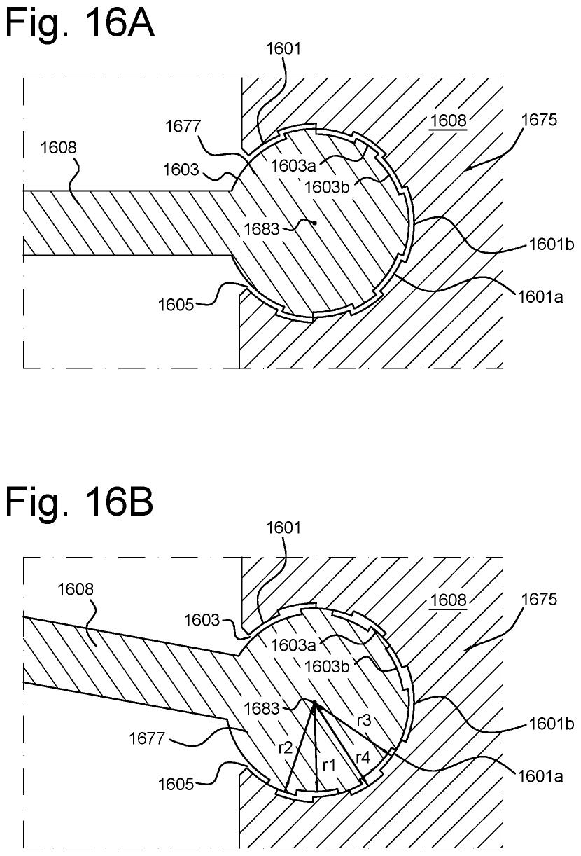

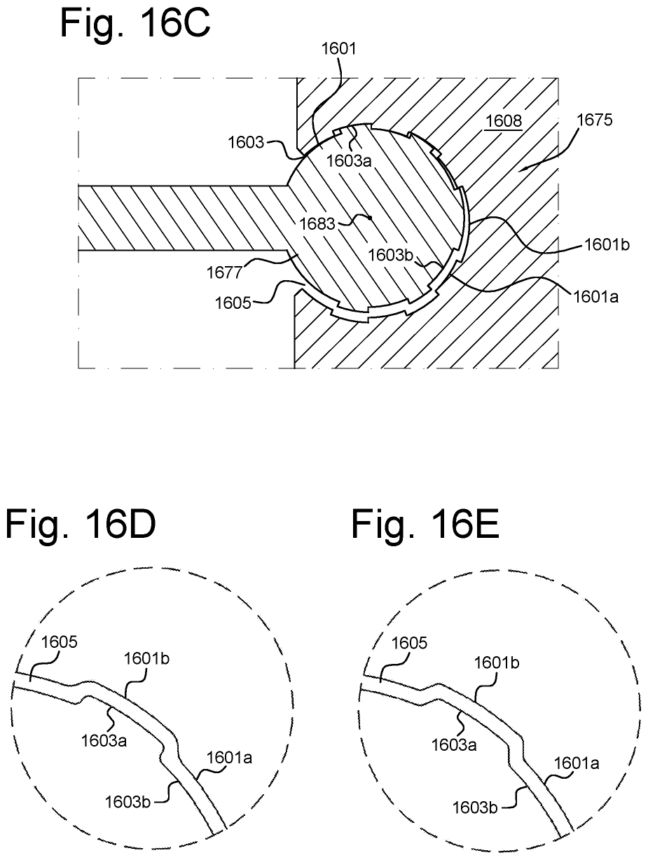

Examples of hinges having the slotted structure as shown in FIG. 13a can be found in e.g. FIG. 16A, and 16E-16H of WO2020/080938 of the present applicant. Similar structures can also be found in U.S. Pat. No. 5,807,241. The present invention addresses play in all such hinges but is not limited to examples of these prior art documents.

Loss of longitudinal response of the steering elements is equal to the number of hinges times the play per hinge as shown in FIG. 13b. FIG. 13b shows how the hinge 1302 is pressed together in its longitudinal direction by a force 1310 indicated to be exerted from the right side to the left side in the figure. However, the longitudinal pressing force may be resulting from other forces too. As a result of the longitudinal pressing force 1310 and due to the play present in the hinge 1302 in a rest condition one or more of the convex portions 1304 and the concave portion 1306 move to one another in the longitudinal direction along a distance equal to the play in one couple of convex portions 1304 and concave portion 1306 until that they will contact one another.

In practice, a long steerable instrument that is used for, for example, gastro intestinal applications can be up to 2 meters long and can have between 200 and 800 hinge segments 1308. A typical play per hinge segment 1308 equals the width of the slot between a couple of a convex portion 1304 and concave portion 1306 and can be around 0.02 mm. So, the total longitudinal play can be as large as 4 to 16 mm. This means that longitudinal steering element activation with less than 4 to 16 mm does not result in steering of the tip at the distal end. Longitudinal play can be minimized by pre-tensioning the longitudinal steering elements. When one assembles the instrument, one can pull all longitudinal steering elements simultaneously and so reduce the longitudinal play in the hinges. By fixating the actuation end of the steering elements in this pre-tension position one has reduced the longitudinal play permanently. However, this can only be done with longitudinal play and not with tangential and radial play.

FIG. 13b clarifies play in the tangential direction. When a tangential force 1312 is exerted on the hinge 1302, a convex portion 1304 and concave portion 1306 in a hinge segment 1308 will rotate relative to one another to an extent depending on the tangential play in the slot between them resulting from the manufacturing process.

For example, again, each slot may be as wide as 0.02 mm, so the total tangential play of all hinges in an instrument with 200 to 800 hinge segment 1308 can also be 4 to 16 mm in one direction, which means that when an instrument has a diameter of for example 4 mm the tangential play between the proximal end and the distal end of the instrument can be approximately 115 degrees to 458 degrees in one tangential direction. Rotating from one ultimate position to the ultimate position in the other direction even has double that play. Also the loss of rotational response is equal to the number of hinge segments 1308 times the play per hinge segment 1308.

An invasive instrument which is provided with longitudinal steering elements 16, 120/120a, 130/130a which result from providing a cylindrical element with a pattern of slots also suffers from play in relation to such longitudinal steering elements 16, 120/120a, 130/130a. For instance, referring to FIG. 3b, even the parts 62, 66 of the longitudinal steering elements 16 are separated from parts of adjacent longitudinal steering elements 16 by a narrow slot which is as small as the width of the (laser) beam used to produce the slots. These slots cause a same play in the tangential direction of the instrument as explained with reference to the hinges shown in FIGS. 13a-13c.

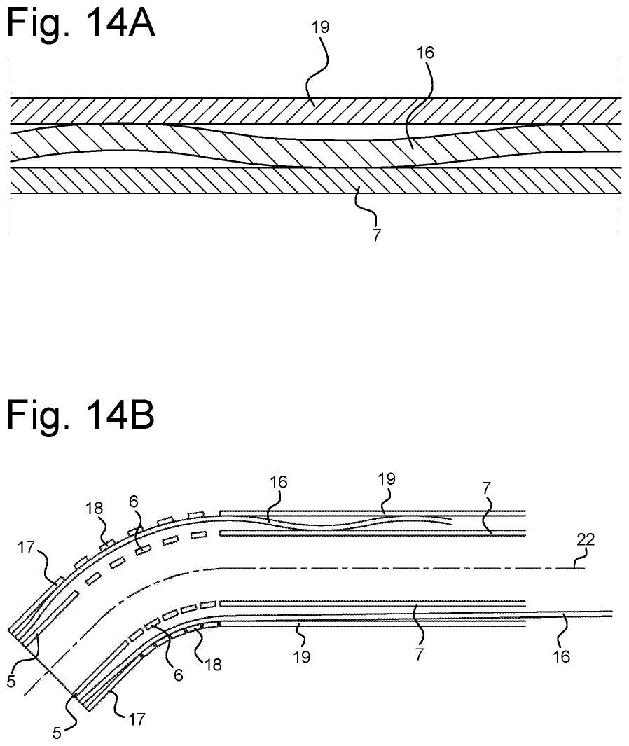

When a longitudinal steering element is used as a pushing steering actuator and the element has tangential play, the element has room to buckle in the tangential direction. When one pushes the longitudinal steering element on the proximal end of the instrument over a certain distance, part of the displacement is lost due to this buckling and only part of the displacement is transferred to the distal end of the instrument. Therefore, the steering response is adversely affected and there will be a hysteresis in steering behavior. Also the buckling creates points of contact and so points where friction is generated between the longitudinal steering element and adjacent elements, which also negatively affects steering of the instrument. This is further explained with reference to FIG. 14a.

FIG. 14a shows portion XIVa of FIG. 2 on an enlarged scale. FIG. 14a shows a longitudinal steering element 16 between two adjacent longitudinal steering elements 16. However, the same problem exists for portions of longitudinal elements 16 (as well as the longitudinal steering elements 120/120a, 130/130a of FIG. 7) between other portions of the cylindrical element which are not longitudinal steering elements themselves. FIG. 14a shows longitudinal steering element 16 in a condition where a pressing force is applied to the longitudinal steering element 16 from one of its ends, in most cases the proximal end, towards the opposing end, in most cases the distal end. The pressing force may result from pushing longitudinal steering element 16 from the proximal end towards the distal end to control bending of a bendable distal part. As a consequence, longitudinal steering element 16 may take the form of a wave shape as seen in the radial direction of the instrument, where the maximum amplitude of the waveform is determined by the distance between the two adjacent longitudinal steering elements 16. This can have adverse effect on the instrument's performance caused by buckling of the longitudinal steering element 16 and ‘pulling out the play’ by longitudinal steering element 16. This results in loss of displacement at the actuated end of the instrument which will adversely affect the steering response, for instance a smaller deflection angle of the tip may be obtained than desired.

However, apart from tangential play, the longitudinal steering elements 16, 120/120a, 130/130a may also exhibit a radial play, as will be explained with reference to FIG. 14b. FIG. 14b shows an enlarged view of a part of the instrument as indicated with XIVb in FIG. 1. The same reference numbers refer to the same components. Reference number 22 refers to a central axis of the instrument.

As explained above, a typical instrument may be made from cylindrical elements 2, 3, 4 that are slid into each other to assemble the instrument. The cylindrical elements 2, 3, 4 must have a certain amount of play to be able to do so. In the cross section view of the distal end as shown in FIG. 14b, the lower shown longitudinal steering element 16 is pulled from the proximal end whereas the upper longitudinal steering element 16 is pushed from the proximal end. Consequently, in the bendable distal portion of the instrument, the lower longitudinal steering element 16 is pushed downward such as to contact flexible part 18 of outer cylindrical element 4. Contrary, in the bendable distal portion of the instrument, upper longitudinal steering element 16 is pushed upward such as to contact flexible part 18 of outer cylindrical element 4.

This can have adverse effect on the instrument's performance too caused by buckling of the longitudinal steering element 16 and ‘pulling out the play’ by longitudinal steering element 16. In this way, also radial play results in loss of displacement at the actuated end of the instrument which will adversely affect the steering response, for instance a smaller deflection angle of the tip may be obtained than desired.

### Solution to Play in Invasive Instruments

In instruments built from separately machined and assembled parts, one can manage play in longitudinal, tangential and radial direction by managing the dimensions and tolerances of the separate parts. Because of the, potentially, strong impact of play on an instrument's performance these parts usually have to be made with narrow tolerances which makes the parts expensive to manufacture and often difficult to assemble correctly. In instruments that are made from tubes out of which the required parts or elements are machined integrally, one does not have the possibility to decrease the play between the parts to a lesser amount than the width of the material removal means. The material removal means can be a laser beam that melts and evaporates material or water jet cutting beam and this beam can have a width of 0.01 to 2.00 mm, more typically for this application, between 0.015 and 0.04 mm.

Extra manufacturing actions have to be used to manage part or element dimensions and the play between them. The invention describes a method that provides for managing play in all aforementioned directions by laser/water cutting hinges, longitudinal steering elements and other features such that the different types of play are set at the required levels.

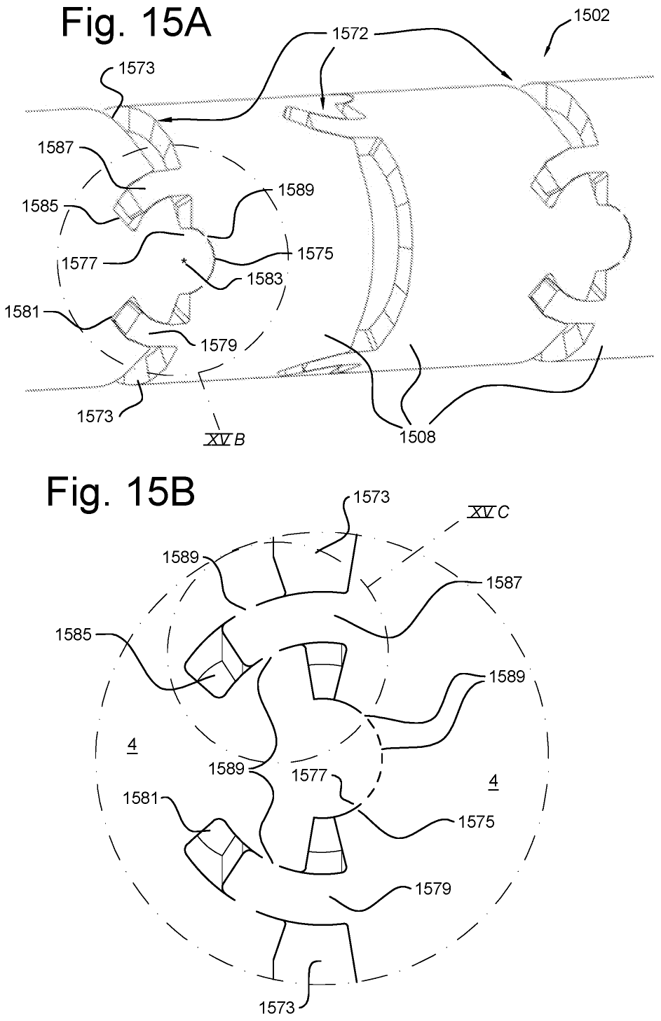

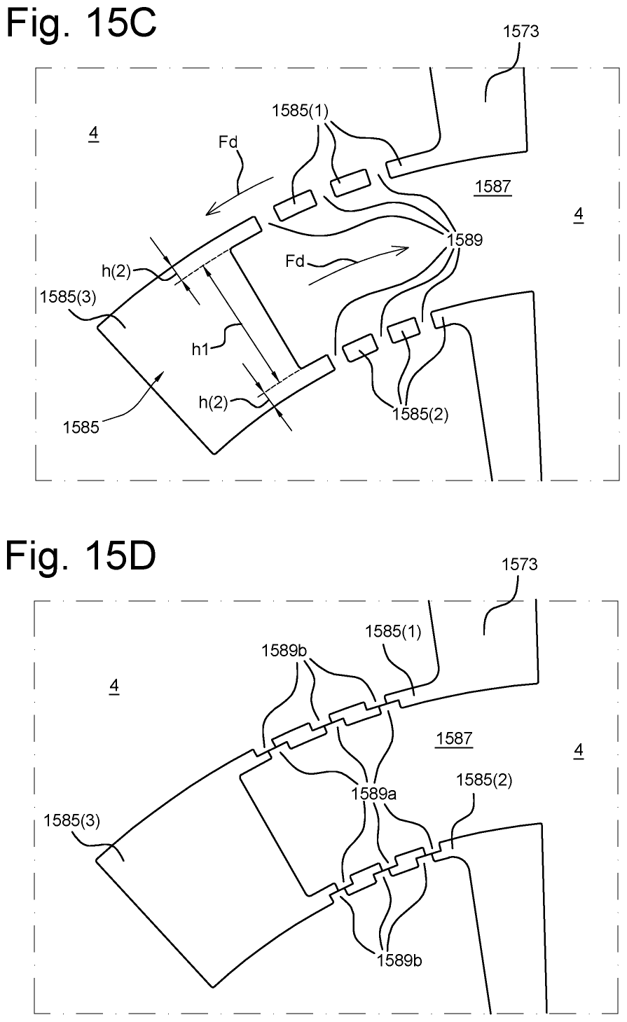

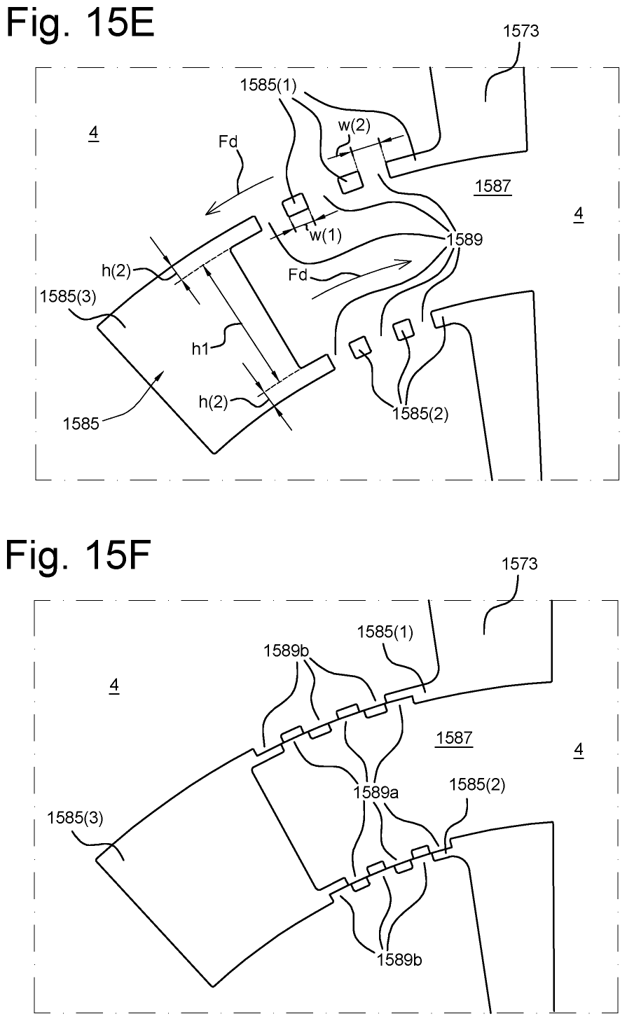

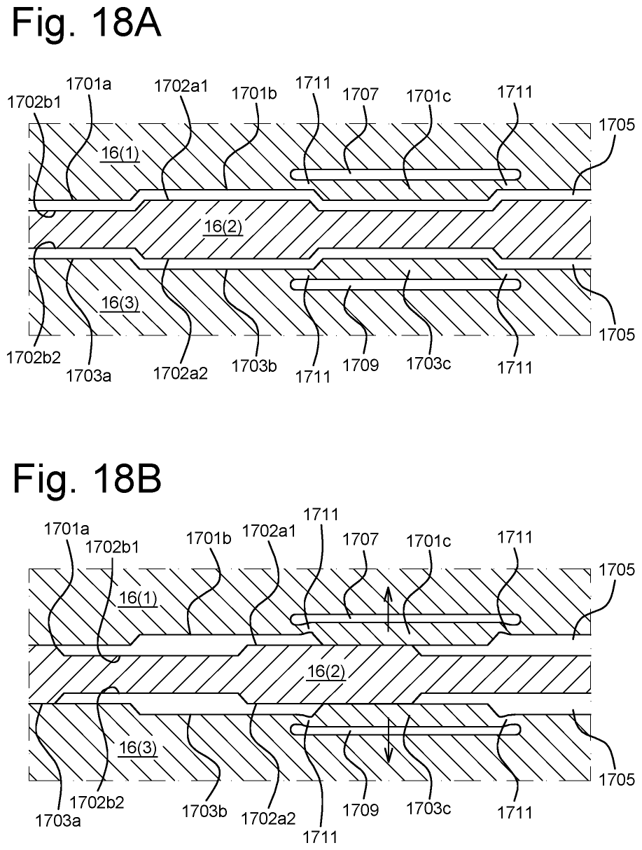

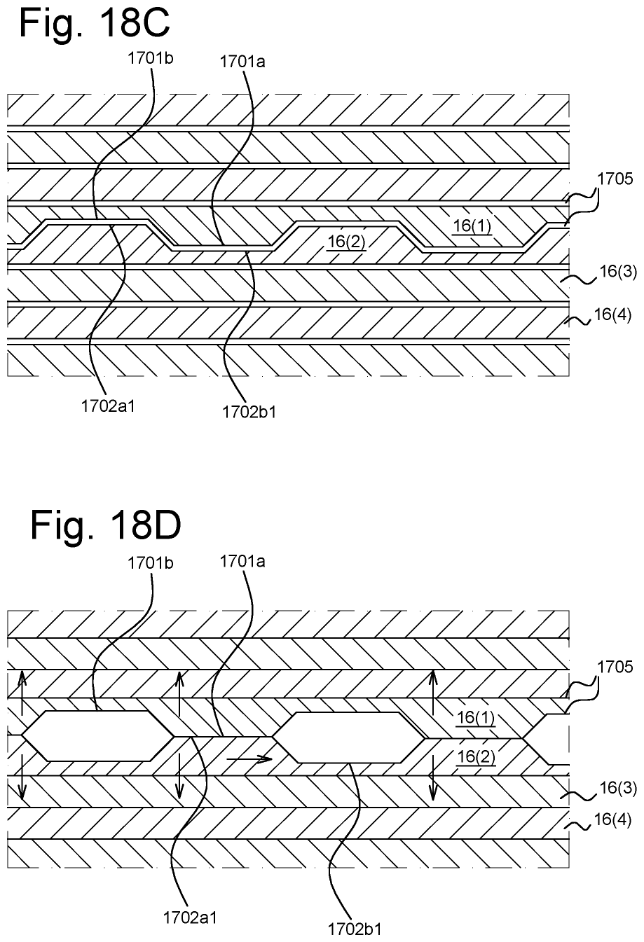

However, before explaining embodiments of the present invention, an overview of a play reducing manufacturing method of hinges is provided as known from FIGS. 16A, 18A-18E from prior art WO2020/080938. They are repeated here as FIGS. 15a-15f. Embodiments of the present invention can be applied in the examples of these figures too.

FIG. 15a shows an example of a hinge 1502 in a cylindrical element. Four adjacent hinge segments 1508 are shown. Hinge 1502 has a slotted structure 1572 which comprises, as shown at the left hand side, a circumferential slot 1573 in the tube element. Slot 1573 extends circumferentially.

Slot 1573 has two opposing side walls both extending circumferentially. Slot 1573 has a curved slot 1585 extending longitudinally, here in the distal direction, from one such side wall and formed as a channel along a portion of a circle having a center point 1583. A lip 1587 that is shaped as a portion of a circle and matches the form of the curved slot 1585 extends from the opposing side wall into this curved slot 1585.

Slot 1573 has a further curved slot 1581 extending longitudinally, here in the distal direction, from one side wall and formed as a channel along a portion of the same circle along which curved slot 1585 extends. A lip 1579 that is shaped as a portion of a circle and matches the form of the curved slot 1581 extends from the opposing side wall into this curved slot 1581.

Symmetrically located between lips 1587, 1579 the slotted structure comprises a convex section 1577 with a circular outside surface that is separated from an oppositely located concave circular section 1575 by a small slot resulting from laser/water cutting. Convex section 1577 and concave section 1575 have matching circular outside surfaces such that convex section 1577 can rotate in concave section 1575 about center point 1583.

At the other side of the cylindrical element 180° rotated away in the tangential direction, the slotted structure has an identical shape with two further lips and mating convex and concave sections. Thus, two hinge segments 1508 of the tube element at either side of the slot 1573 can “rotate” relative to one another about two center points 1583, such that they deflect relative to one another. The lips 1579, 1587 move in the curved slots 1581, 1585 during such rotation and provide no extra friction. The lips 1579, 1587 provide extra tangential stability to the tube element when one rotates the entire tube element about its longitudinal central axis. This is an important aid in increasing torque stiffness. They define a predetermined tangential play as determined by the width of the slots 1581, 1585 surrounding the lips 1579, 1587.

Hereinafter, a cylindrical element including two adjacent hinge segments 1508 such as to allow bending of the cylindrical element about the hinge segments 1508 will be explained in more detail. The slotted structure allows opposite cylindrical element portions of the hinge to bend to a predetermined maximum angle.

Immediately after the cutting process, opposing hinge segments 1508 of the hinge at either side of the slot 1573 are still attached to one another by one or more fracture elements 1589 designed such as to break once two opposing hinge segments 1508 are rotated relative to one another.

As shown in FIG. 15a, slot 1573 between convex section 1577 and concave section 1575 is interrupted one or more times such that convex section 1577 and concave section 1575 are connected to one another by one or more fracture elements in the form of small bridges 1589. These small bridges 1589 operate as “fracture elements” as will be explained in more detail with reference to FIG. 15b-15d. I.e., these fracture elements 1589 are made on purpose when the instrument is manufactured but are so weak that they will break once convex section 1577 is rotated relative to concave section 1575 with a predetermined force. Before breaking, the fracture elements 1589 provide the cylindrical element with a predetermined extra stiffness such that the cylindrical element can be maneuvered more easily when inserting the cylindrical element inside an other cylindrical element or inserting an other cylindrical element in the cylindrical element. Once broken, the fracture elements 1589 play no role anymore and convex section 1577 can freely rotate in concave section 1575.

At a predetermined longitudinal distance away from slots 1573, the cylindrical element comprises an identical slot but then rotated 90° in the tangential direction relative to the slot 1573. Thus, two further points of rotation are provided at said predetermined longitudinal distance about which the cylindrical element can rotate but then in a direction perpendicular to the direction of rotation allowed by center points 1583.

At a further predetermined longitudinal distance away from slot 1573, the structure as defined by slot 1573 is again repeated but now identical to the one formed by slot 1573. These alternating structures are repeated several times in the longitudinal direction. Thus, the cylindrical element comprises 90° tangentially rotated centers of rotation at predetermined longitudinal distances away from each other allowing the cylindrical element to deflect in all directions.

FIG. 15b shows how slot 1573 in, for example, outer cylindrical element 4 at the location where convex section 1577 is located inside concave section 1575 can be made narrow while using fracture elements 1589. FIG. 15b shows an enlarged portion of the slotted structure 1572 shown in FIG. 15a directly after its manufacturing. It shows that convex section 1577 is, then, still attached to concave section 1575 by means of a plurality of fracture elements 1589. Moreover, lips 1587 and 1579 are still attached to the opposing part of the cylindrical element 4 by means of one or more fracture elements 1589.

Such fracture elements 1589 can be made as follows. Slot 1573 is, e.g., made by directing a laser beam, or water beam, with a predetermined energy and width to the cylindrical element such as to cut through the entire thickness of the cylindrical element. The laser beam moves relative to the cylindrical element outer surface, e.g., by moving a laser source relative to that outer surface. However, at locations where fracture elements 1589 are to be formed, the laser beam is interrupted for a certain period of time whereas the laser source still moves relative to the cylindrical element outer surface.

As explained above, when deflecting different portions of slotted structure 1572 relative to one another for the first time, these fracture elements 1589 will fracture. A great advantage of such fracture elements 1589 is that, after being fractured, the distance between two opposite sides of the fracture element 1589 is (much smaller) than the width of the slot which results from the laser cutting. This distance may be said to be substantially 0 μm which results in an extremely low play between opposing parts. Consequently, play between convex section 1577 and concave section 151575 is reduced.

FIGS. 15c and 15d shows such fracture elements 1589 of a first embodiment in greater detail. I.e., FIG. 15c is an enlarged view of a portion XVc shown in FIG. 15b. Curved slot 1585 is shown to have three portions 1585(1), 1585(2), and 1585(3). Together these three portions 1585(1), 1585(2), and 1585(3) form a U shaped channel where portions 1585(1) and 1585(2) form the long sides and portion 1585(3) forms the short, lower base side of the U shaped channel. Lip 1587 is surrounded by portions 1585(1), 1585(2), and 1585(3).

Like slot 1573 portions 1585(1), 1585(2), and 1585(3) are formed by, e.g., laser or water cutting through cylindrical element 4. The width h(2) of portions 1585(1) and 1585(2) may be the same and be substantially equal to the width of the laser (or water) beam used to produce these portions 1585(1), 1585(2). The size of portion 1585(3) depends on the path length lip 1587 should be able to move within curved slot 1585. Directly after such cutting action, lip 1587 is still attached to opposing portions of cylindrical element 4 by means of the fracture elements 1589. As explained above, that provides cylindrical element 4 with more rigidity after the cutting process such that cylindrical element 4 can be treated easier, e.g. when another cylindrical element is inserted into cylindrical element 4 or cylindrical element 4 is inserted into another cylindrical element.

In use, the slotted structure shown in FIGS. 15b-15d is part of a hinge 1502, as explained above. If the portion of the cylindrical element 4 in which the slotted structure is located is bent, a force Fd is exerted by means of which lip 1587 is moved outside curved slot 1585. The actual force Fd may be in a direction opposite the one shown in FIG. 15c such that lip 1587 is moved inside curved slot 1585. Due to force Fd caused by bending cylindrical element 4 fracture elements 1589 will fracture such that lip 1587 can move freely inside curved slot 1585.

FIG. 15d shows that each one of the fracture elements 1589 will fracture into two opposing separated fracture element portions 1589a and 1589b. In an embodiment, each fracture element 1589 has a predetermined width and the fracture element portions 1589a, 1589b will have substantially the same width at their outer surfaces facing one another. Thus, in use, these fracture element portion 1589a, 1589b will be in contact with one another with their outer surfaces facing one another as long as the movement of the fracture element portions 1589a, 1589b relative to one another is not larger than this width. In an advantages embodiment, the width is so large that even in their maximum possible relative movement, as allowed by the width of slot 1573, fracture element portions 1589a, 1589b still contact each other. So, tangential play in the slotted structure is kept to a minimum.

FIGS. 15e and 15f show a further embodiment of fracture elements 1589. Fracture elements 1589 of FIGS. 15e and 15f may have the same form as in FIGS. 15c and 15d, However, the distance w(1) between adjacent fracture elements 1589 is now smaller than the width w(2) of the fracture elements 1589 themselves. In FIGS. 15c and 15d the situation is shown where this mutual distance between adjacent fracture elements 1589 is larger than the width of the individual fracture elements 1589. Consequently, in the embodiment of FIGS. 15e and 15f, even when lip 1587 and opposing side of cylindrical element 4 are moved relative to one another along a distance larger than a distance equal to width w(1) (cf. FIG. 15f), one or more of the fracture element portions 1589a, 1589b may still contact one another because they cannot move inside the space between adjacent fracture elements 1589. I.e., that space is too small to accommodate such fracture elements 1589. This provides an even larger playless capacity in the tangential direction.

Referring back to FIG. 15b, fracture elements 1589 between convex section 1577 and concave section 1575 are designed in the same way. So, by rotating convex section 1577 inside concave section 1575 with a predetermined force fracture elements 1589 will fracture and each fracture element 1589 leaves two fracture element portions 1589a and 1589b. These latter fracture element portions 1589a and 1589b will have the same form and function as shown in FIG. 15d. I.e., the slotted structure is configured such that convex section 1577 can rotate within concave section 1575 until rotation is blocked by the structure. The fracture elements 1589 have such a width that, after being fractured, fracture element portions 1589a and 1589b have surfaces facing one another and always contacting one another during the entire maximum possible rotation. As can be seen, consequently, even after being manufactured convex section 1577 and concave section 1575 contact one another such that play in the longitudinal direction between convex section 1577 and concave section 1575 is kept to a minimum.

The more slotted structures of the flexible cylindrical element 4, forming the hinge segments 1508, are produced with such fracture elements 1589 the more hinge segments 1508 will show playless properties both in the tangential and longitudinal direction. Consequently, a flexible cylindrical element 4 can be made in which play in both the tangential and longitudinal direction is drastically reduced which is especially an advantages feature for longer instruments, e.g., instruments longer than 1 meter.

Fracture elements 1589 should be designed in the following way. Before being fractured, each fracture element 1589 is attached to opposite portions of cylindrical element 4. These opposite portions of cylindrical element 4 have a geometrical shape such that the stresses in the fracture element 1589 are higher than in the surrounding material and/or structure. Therefore, if a deflection or a high enough force is applied on a structure with a fracture element 1589 the stress in the fracture element rises above the yield stress of the tube material, causing permanent deflection of fracture element 1589. Applying even more deflection or a higher force results in the stress reaching the ultimate tensile stress causing a fracture of fracture element 1589. An other mechanism to break the fracture element is achieved by applying low or high cycle fatigue to fracture element 1589. The stress in fracture element 1589 is raised above the fatigue limit, causing a fatigue fracture. In all cases the stresses in the surrounding structure/material stays at least below the yield stress of the tube material.

FIGS. 16a and 16b show an embodiment of a hinge with two opposing hinge segments 1608 in which play is reduced in accordance with the present invention. FIGS. 16a and 16b are only very schematic. They can relate to the hinge 1502 of FIGS. 15a-15f or any other type of hinge cut in a cylindrical element having a hinge segment 1608 with a convex portion 1677 and a hinge segment 1608 with a concave portion 1675, wherein the convex portion 1677 is arranged and configured to be rotated inside concave portion 1675. The outside edge 1603 of convex portion 1677 is serrated and so is the outside edge 1601 of concave portion 1675.

The serrated outside edge 1603 of convex portion 1677 has extending portions 1603a and an indented portion 1603b between each two adjacent extending portions 1603a. Both the extending portions 1603a and the indented portions 1603b may have a circular form extending along a circle about center point 1683. However, they may have any other suitable form. In the shown embodiment, the indented portions 1603b extend along a first circle having a first radius r1. The extending portions 1603a extend along a second circle having a second radius r2 which is larger than the first radius r1.

The serrated outside edge 1601 of concave portion 1675 has extending portions 1601a and an indented portion 1601b between each two adjacent extending portions 1601a. Both the extending portions 1601a and the indented portions 1601b may have a circular form extending along a circle about center point 1683. However, they may have any other suitable form. In the shown embodiment, the extending portions 1601a extend along a third circle having a third radius r3. The indented portions 1601b extend along a fourth circle having a fourth radius r4 which is larger than the third radius r3.

FIG. 16a shows the hinge in its status directly after it has been manufactured and not yet used in any way. For the purpose of this document, this is called the “manufactured state” of the hinge. The serrated outside edge 1603 of convex portion 1677 and the serrated outside edge 1601 of the concave portion 1675 are separated from one another by a slot 1605 which results from (laser) cutting both hinge segments 1608 from a cylindrical element. This slot 1605 may have a constant width along its entire length, e.g., for medical applications in a range between 0.01 to 2.00 mm, more typically for this application, between 0.015 and 0.04 mm.

In an embodiment, the second radius r2 is about equal to the third radius r3. I.e., they may be equal within manufacturing tolerances which may be less than 10%, preferably less than 5% of r2 or r3. In the shown embodiment, the second radius r2 is not larger than the third radius r3. The reason therefor will become apparent from the description of FIG. 16b. An alternative definition is that the extending portions 1603a have a height which is at maximum about equal to the width of slot 1605 (or distance) between an adjacent indented portion 1603b and an opposing extending portion 1601a, where “about equal” again refers to equal within manufacturing tolerances, i.e., that height and distance differ by 10% or less, alternatively 5% or less, or further alternatively 1% or less.

FIG. 16a shows convex portion 1677 and concave portion 1675 in the manufactured state when they have not been rotated relative to one another. FIG. 16b shows convex portion 1677 and concave portion 1675 in a state wherein convex portion 1677 and concave portion 1675 have been rotated relative to one another about center point 1683. In the shown embodiment, the second radius r2 and third radius r3 are about equal such that, when convex portion 1677 and concave portion 1675 rotate relative to one another, an extending portion 1603a of convex portion 1677 abuts an extending portion 1601a of concave portion 1675. In case convex portion 1677 has a plurality of extending portions 1603a distributed along its outer edge 1603 and concave portion 1675 has a plurality of extending portions 1601a distributed along its outer edge 1601, several of the extending portions 1603a may abut several of the extending portions 1601a. Depending on the exact design, several of the extending portions 1603a may abut several of the extending portions 1601a along a circular arc of a degrees about center point 1683 where a may be >45 degrees but a may alternatively be >180 degrees (as in FIGS. 16a and 16b).

At locations where one or more extending portions 1603a of convex portion 1677 abut one or more extending portions 1601a of concave portion 1675 they cannot move any more towards one another in the radial direction as seen from center point 1683. So, in the rotated status, play between abutting extending portions 1603a and extending portions 1601a is removed. Depending on the design, play may have been removed in at least one of the longitudinal direction or tangential direction of the cylindrical element in which the hinge is made.

Outer edge 1603 of convex portion 1677 has a transition edge portion between each extending portion 1603a and each adjacent indented portion 1603b. Outer edge 1601 of concave portion 1675 has a transition edge portion between each extending portion 1601a and each adjacent indented portion 1601b. Transition edge portions of outer edge 1603 and transition edge portions of outer edge 1601 are separated from one another by a distance which, after manufacturing, is as wide as the width of slot 1605 resulting from the cutting process. The width of slot 1605 at locations between opposing transition edge portions of outer edge 1603 and transition edge portions of outer edge 1601 may be as wide as the width of slot 1605 at locations between other opposing portions of outer edges 1603 and outer edge 1601 but that is not necessary.

If the width of slot 1605 at locations between opposing transition edge portions of outer edge 1603 and transition edge portions of outer edge 1601 is very small relative to the radius of convex portion 1677, only a very small rotation between convex portion 1677 and concave portion 1675 will result in extending portions 1603a of convex portion 1677 abutting extending portions 1601a of concave portion 1675. Consequently, only when two adjacent hinge segments 1608 are longitudinally aligned, i.e. the status equal to the manufactured status, they show some play relative to one another which is as large as the width of slot 1605. In a relative rotated (or deflected) status about a certain deflection angle β, however, all play may be removed. In a typical example, such deflection angle β may be <5 degrees or even <3 degrees or <1 degrees. In use, many adjacent hinge segments of the invasive instrument may be deflected relative to one another about an angle>β, e.g. due to curvatures in the canal, for instance a human intestinal canal, in which they are inserted. So, in use, a high percentage of play in hinges may be reduced by the embodiment of FIGS. 16a and 16b.

If the extending portions 1603a have a height which is smaller than the width of slot 1605 between an adjacent indented portion 1603b and an opposing extending portion 1601a, some play will remain in the rotated state of the hinge.

If the extending portions 1603a have a height which is larger than the width of slot 1605 between an adjacent indented portion 1603b and an opposing extending portion 1601a, the hinge cannot rotate or rotates with severe friction. So, this should be avoided.

To prevent that transition edge portions of outside edge 1603 and opposing transition edge portions of outside edge 1601 get caught behind one another when one tries to rotate the hinge, cf. FIG. 16c, in an embodiment, these transition edge portions of outside edge 1603 and transition edge portions of outside edge 1601 are not parallel to a line through center point 1683 and the transition edge portion but are angled relative to that line, i.e., they are chamfered, such that the extending portions 1603a and 1601a have a tapered form. This is shown in FIGS. 16d, 16e. The outward angle between the transition edge portion and the indented portion 1603 is an obtuse angle. Then, extending portion 1603a can be easily moved to a position at least partly opposite extending portion 1601a.

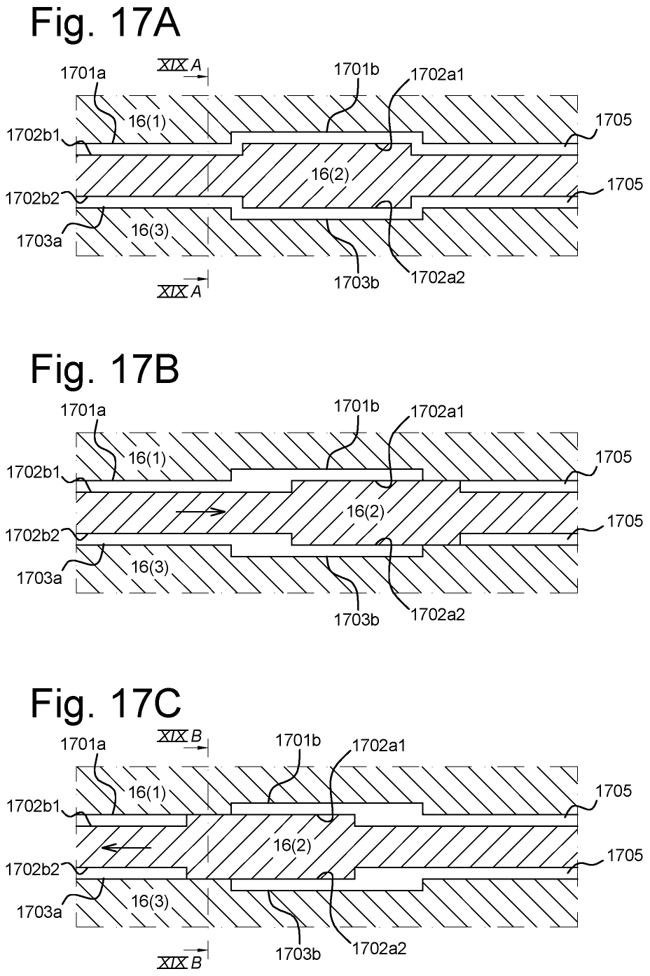

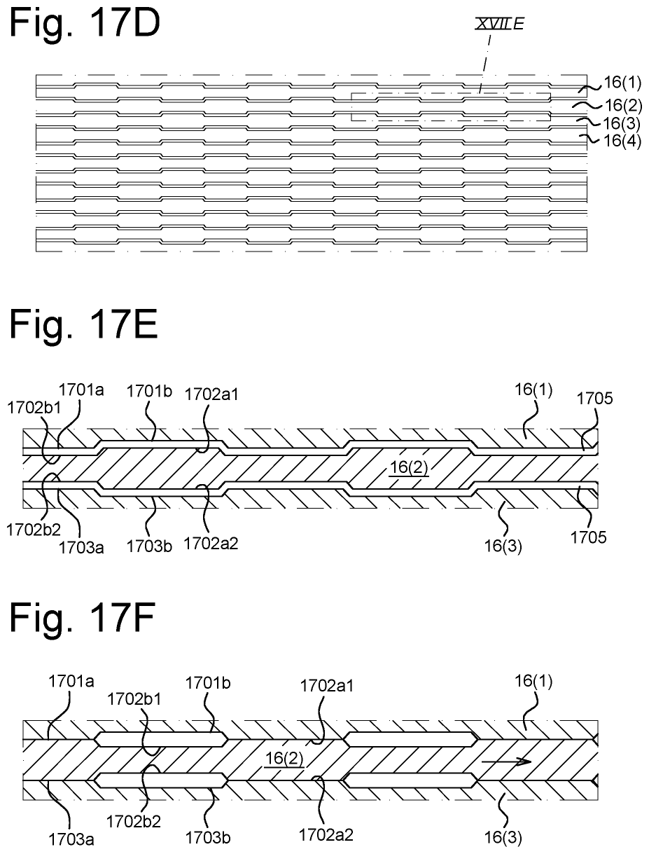

FIGS. 17a-17c show how similar principles can be applied between adjacent longitudinal elements. Each one of the FIGS. 17a-17c show three adjacent longitudinal elements 16(1), 16(2), 16(3). However, either one of the longitudinal elements 16(1) and 16(3) can be substituted by a portion of the cylindrical element which is not movable in the longitudinal direction of the instrument, such that only longitudinal element 16(2) is movable in the longitudinal direction. Moreover, there may be more than three adjacent longitudinal elements. FIG. 17a shows the status directly after the elements have been manufactured and not yet used in any way. For the purpose of this document, this is called the “manufactured state” of the elements relative to one another. FIGS. 17b and 17b show statuses after relative displacements.

Directly after the manufacturing process, there is a slot 1705 between longitudinal element 16(2) and longitudinal element 16(1), as well as between longitudinal element 16(2) and longitudinal element 16(3). The slots 1705 at either side of longitudinal element 16(2) may have the same dimensions but they may also be different. Either one may have a constant width but that is not necessary.

In the status of FIG. 17a, which corresponds to the status directly after manufacturing, at one longitudinal side, longitudinal element 16(2) has at least one extending portion 1702a1 extending towards an indented portion 1701b of longitudinal element 16(1). Adjacent to extending portion 1702a1, longitudinal element 16(2) comprises an indented portion 1702b1 opposite an extending portion 1701a of longitudinal element 16(1). Longitudinal element 16(2) may be provided with such an indented portion 1702b1 at either side of extending portion 1702a1. Likewise, longitudinal element 16(1) may be provided with an extending portion 1701a at either side of indented portion 1701b. In fact, the shown structure may be repeated along the longitudinal direction of the instrument.

Also in the status of FIG. 17a, at its other longitudinal side, longitudinal element 16(2) has at least one extending portion 1702a2 extending towards an indented portion 1703b of longitudinal element 16(3). Adjacent to extending portion 1702a2, longitudinal element 16(2) comprises an indented portion 1702b2 opposite an extending portion 1703a of longitudinal element 16(3). Longitudinal element 16(2) may be provided with such an indented portion 1702b2 at either side of extending portion 1702a2. Likewise, longitudinal element 16(3) may be provided with an extending portion 1703a at either side of indented portion 1703b.

Extending portions 1702a1 and 1702a2 have respective heights which are, in an embodiment, equal to or smaller than the width of the respective surrounding slots 1705.

In the status of FIG. 17a, longitudinal elements 16(1), 16(2), 16(3) show mutual play equal to the width of the respective slots 1705 in between them. In a cylindrical instrument wherein these longitudinal elements 16(1), 16(2) and 16(3) are cut from the material of a cylindrical element, this mutual play is a tangential play.

Again, in medical applications, the instrument may have a diameter of e.g. 4 mm, and the slot 1705 may have a width of 0.01 to 2.00 mm, more typically for this application, between 0.015 and 0.04 mm. In long instruments, e.g., longer than 1 m, the longitudinal elements 16(1), 16(2), 16(3) may also be longer than 1 m. In such instruments, this slot width may have a substantial influence on the total tangential play of the instrument when the proximal end is rotated in the tangential direction relative to the distal end. This may affect the responsiveness of the instrument to a too high extent. Also, and more importantly, the tangential play gives room for the longitudinal element to buckle under compression load. So as was explained earlier, this room for buckling can adversely affect steering response.

Though FIGS. 17a-17c show indented portions and extending portions in all longitudinal elements 16(1), 16(2), 16(3), in an embodiment, they may only be applied in longitudinal elements 16(1) and the longitudinal side of longitudinal element 16(2) opposing longitudinal element 16(1). Then, longitudinal element 16(3) may have a straight longitudinal side and the longitudinal side of longitudinal element 16(2) opposing longitudinal element 16(3) may also be straight.

Moreover, the length of extending portions 1702a1 and 1702a2 may be different. Moreover, extending portions 1702a1 and 1702a2 may be located at different longitudinal locations along the instrument.

FIG. 17b shows the status of the three longitudinal elements 16(1), 16(2), 16(3) relative to one another after longitudinal element 16(2) has been relatively shifted sideways to the right hand side, as indicated with an arrow. In case extending portions 1702a1 and 1702a2 have respective heights which are equal to the width of the respective surrounding slots 1705 when longitudinal element 16(2) is shifted to the right along a distance larger than the width of slot 1705, extending portion 1702a1 abuts opposing extending portion 1701a, at least partially, and extending portion 1702a2 abuts opposing extending portion 1703a, at least partially. So, the mutual distance between the three longitudinal elements 16(1), 16(2), 16(3) is than reduced to 0 (zero).

FIG. 17c shows the status of the three longitudinal elements 16(1), 16(2), 16(3) relative to one another after longitudinal element 16(2) has been relatively shifted sideways to the left hand side, as indicated with an arrow. In case extending portions 1702a1 and 1702a2 have respective heights which are equal to the width of the respective surrounding slots 1705 when longitudinal element 16(2) is shifted to the left along a distance larger than the width of slot 1705, extending portion 1702a1 abuts opposing extending portion 1701a, at least partially, and extending portion 1702a2 abuts opposing extending portion 1703a, at least partially. So, the mutual distance between the three longitudinal elements 16(1), 16(2), 16(3) is than reduced to 0 (zero).

In the shown embodiment, longitudinal element 16(2) has transition edge portions between each extending portion 1702a1 and the adjacent indented portions 1702b1. Likewise, longitudinal element 16(1) has transition edge portions between each indented portion 1701b and the adjacent extending portions 1701a. Transition edge portions of longitudinal elements 16(1) and 16(2) are separated by a distance as large as the width of the slot 1705 between them. If that width is very small, e.g., as indicated above between 0.01 and 2.00 mm, more typically for this application, between 0.015 and 0.04 mm, adjacent longitudinal elements need only shift in the longitudinal direction to a small extend to arrive at the situation of FIG. 17b or 17c, wherein tangential play is reduced or even eliminated.

The same applies to the opposing longitudinal sides of longitudinal elements 16(2) and 16(3). I.e., longitudinal element 16(2) has transition edge portions between each extending portion 1702a2 and the adjacent indented portions 1702b2. Likewise, longitudinal element 16(3) has transition edge portions between each indented portion 1703b and the adjacent extending portions 1703a. Transition edge portions of longitudinal elements 16(2) and 16(3) are separated by a distance as large as the width of the slot 1705 between them. If that width is very small, e.g., as indicated above between 0.01 and 2.00 mm, more typically for this application, between 0.015 and 0.04 mm, adjacent longitudinal elements need only shift in the longitudinal direction to a small extend to arrive at the situation of FIG. 17b or 17c, wherein tangential play is reduced or even eliminated.

In the embodiment of FIGS. 17a, 17b, the transition edge portions between extending portions and indented portions is shown to be perpendicular to the longitudinal direction of longitudinal elements 16(1), 16(2), 16(3), so in the tangential direction of the instrument. However, these transition edge portions can be angled relative to the longitudinal direction, as shown in FIGS. 17d-17f such that the angle between a transition edge portion and an extending portion to which it is attached is >90°, but, e.g., <135°. In such an embodiment, opposing transition edge portions do not block adjacent longitudinal elements from shifting more in the longitudinal direction than along a distance more than the slot width between opposing transition edge portions because opposing transition edge portions can slide along each other more easily.

FIG. 17d shows the status directly after the elements have been manufactured and not yet been used in any way. For the purpose of this document, this is called the “manufactured state” of the elements relative to one another. FIG. 17f shows a status after relative displacements.

FIG. 17e shows an enlarged view of a detail of FIG. 17d which show a plurality of adjacent longitudinal elements 16(i) (i=1, 2, . . . , l). Note that FIG. 17d is a flattened view of a cylindrical element. FIG. 17f shows the three adjacent longitudinal elements 16(1), 16(2), 16(3) as shown in FIG. 17e but than once the middle longitudinal element 16(2) has been relatively shifted sideways to the right, as indicated with an arrow. Suppose elements 16(1) and 16(3) etc. are longitudinal steering elements and elements 16(2) and 16(4) etc. are stationary elements relative to the body of the instrument, one can permanently reduce tangential play by moving all stationary elements one time in one direction and then fix them to surrounding structure of the body before finally finishing the instrument.

In an embodiment, the height of extending portion 1702a1 of longitudinal element 16(2) may be larger than the width of the slot 1705 between indented portion 1702b1 and opposing extending portion 1701a.

FIGS. 18a-18d show embodiments with negative play. This will be explained hereinafter. FIGS. 18a and 18c show the status directly after the elements have been manufactured and not yet been used in any way. For the purpose of this document, this is called the “manufactured state” of the elements relative to one another. FIGS. 18b and 18d shows statuses after relative displacements.

FIGS. 18a and 18b show a further embodiment of three adjacent longitudinal elements 16(1), 16(2), 16(3) in which play is reduced. In this embodiment, longitudinal element 16(1) has at least on spring portion. Here, the spring portion is implemented by a slot or opening 1707 in longitudinal element 16(1) some distance away from one of the extending portions 1701a in the tangential direction, such that the extending portion becomes a resilient spring portion 1701c attached to the remainder of the longitudinal element 16(1) by means of flexible bridges 1711. Due to the flexible bridges 1711, spring portion 1701c is able to flexibly move in the tangential direction relative to the remainder of the longitudinal element 16(1). In this embodiment, the height of extending portion 1702a1 is larger than the width of slot 1705 between resilient spring portion 1701c and opposing indented portion 1702b1. This is called negative play.

In this embodiment, also longitudinal element 16(3) has at least on spring portion. Here, the spring portion is implemented by a slot or opening 1709 in longitudinal element 16(3) some distance away from one of the extending portions 1703a in the tangential direction, such that the extending portion becomes a resilient spring portion 1703c attached to the remainder of the longitudinal element 16(3) by means of flexible bridges 1711. Due to the flexible bridges 1711, spring portion 1703c is able to flexibly move in the tangential direction relative to the remainder of the longitudinal element 16(3). In this embodiment, the height of extending portion 1702a2 is larger than the width of slot 1705 between resilient spring portion 1703c and opposing indented portion 1702b2. This is also called negative play.

In FIGS. 18a and 18b, the shown embodiment has angled transition edge portions between extending and indented portions like in the embodiment of FIGS. 17c-17e. The outward angle between the transition edge portion and the indented portion 1603 is an obtuse angle. This makes shifting adjacent longitudinal elements 16(2) relative to longitudinal element 16(1) and/or longitudinal element 16(3) easier, as explained above. FIG. 18b shows that longitudinal element 16(2) is relatively shifted sideways to the right relative to both adjacent longitudinal elements 16(1), 16(3). In such a situation, extending portions 1702a1 and 1702a2. Due to the height of extending portion 1702a1 being larger than the width of slot 1705 between spring portion 1701c and opposing indented portion 1702b1 and the height of extending portion 1702a2 being larger than the width of slot 1705 between spring portion 1703c and opposing indented portion 1702b2, both spring portions 1701c and 1703c, respectively, are pushed away by extending portions 1702a1 and 1702a2, respectively, from longitudinal element 16(2) in the tangential direction. By doing so, tangential play between longitudinal elements 16(1), 16(2) and 16(3) is reduced to zero in all cases and this embodiment is not sensitive for manufacturing tolerances. The spring force is designed such that side forces due to normal use of the instrument can be withstood, for example side forces due to the tendency of buckling, but the spring force is engineered such that friction forces are kept to a minimum or even at a pre-engineered value. If in the status of FIG. 18b slots 1707, 1709 are not entirely closed in the tangential direction, some flexible play will remain.

FIGS. 18c and 18d show an embodiment which is a variant to the one shown in FIGS. 18a and 18b. The embodiment of FIGS. 18c and 18d do not have extending portions designed as spring portions but show two adjacent longitudinal elements 16(1) and 16(2) where longitudinal element 16(1) has one or more extending portions 1701a each one opposing an indented portion 1702b1 of longitudinal element 16(2). Moreover, longitudinal element 16(2) may have one or more extending portions 1702a1 each one opposing an indented portion 1701b of longitudinal element 16(1). There may be more longitudinal elements 16(3), 16(4) at either side of the couple of longitudinal elements 16(1), 16(2). Moreover, the structure with extending portions and indented portions may be applied in more adjacent longitudinal sides of other adjacent longitudinal elements as well.

FIG. 18c shows the status directly after manufacturing wherein adjacent longitudinal elements 16(1)-16(4) are separated by a slot 1705 which may have a constant width along its entire length. However, this need not be the case.

Extending portion 1701a has a larger height than the width of slot 1705 between an adjacent couple of an extending portion 1701a1 and an opposing indented portion 1701b. This is called negative play. Similarly, extending portion 1701a1 has a larger height than the width of slot 1705 between an adjacent couple of an extending portion 1701a and an opposing indented portion 1702b2. This is another example of negative play. In the shown embodiment, both extending portions 1701a and 1701a1 have angled transition edge portions to adjacent indented portions 1701b and 1701b1, in the same way as defined for the embodiment of FIGS. 17d-17f.

FIG. 18d shows the status of the embodiment when longitudinal element 16(2) and 16(1) have been relatively shifted sideways to one another in the longitudinal direction as indicated with a horizontal arrow. In that status, extending portions 1701a and 1701a1 abut one another at least partially in the longitudinal direction. Because of the heights of these extending portions 1701a and 1701a1 in relation to the width of the surrounding slot 1705 they are pushed against one another and will exert a force on longitudinal elements 16(1) and 16(2) in opposite tangential directions as indicated with arrows directed in the tangential direction. Because of that, longitudinal elements 16(1) and 16(2) exert a tangential force on adjacent longitudinal elements which, in turn, may then be pushed against their neighboring longitudinal elements. Consequently, all play between adjacent longitudinal elements may be removed in that longitudinal location in the cylindrical element concerned by just moving and fixing one longitudinal element 16(1) or 16(2).

The effect shown in FIG. 18d may even be so strong that all longitudinal element are pushed against one another so strongly that they are clamped and very difficult to be moved longitudinally. This may result in a locking of the instrument in an orientation it had before longitudinal elements 16(1) and 16(2) were longitudinally shifted relative to one another.

The embodiment shown in FIGS. 18c and 18d has eight adjacent longitudinal elements. However, the cylindrical element in which these longitudinal elements are located may have less or more of such longitudinal elements. Moreover, one or more of them may be substituted by another portion of the cylindrical element, like a portion fixed to another adjacent cylindrical element or a spacer which may be floating or fixed to another portion of the cylindrical element.

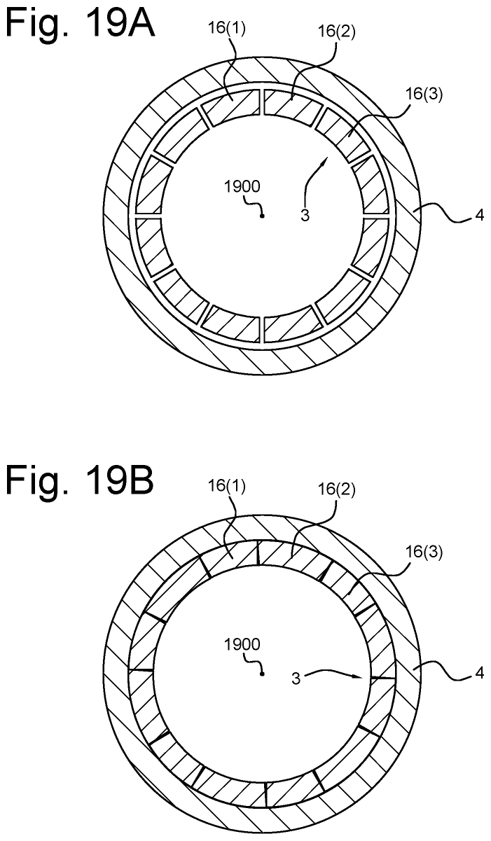

One could also use the creation of negative play between longitudinal elements or between longitudinal elements and a guiding element to reduce radial play. When the longitudinal elements are for example pulled or pushed between guiding elements with negative play, this forces the guiding elements and longitudinal elements radially outwards. An example is shown in FIGS. 19a, 19b. FIG. 19a shows the embodiment in its manufactured state.

FIGS. 19a, 19b show cross sections through an instrument with two cylindrical elements, i.e., intermediate cylindrical element 3 surrounded by outer cylindrical element 4. Intermediate cylindrical element 3 is shown to have a plurality of longitudinal elements of which three adjacent ones are indicated with reference signs 16(1), 16(2), 16(3).

In the situation of FIG. 19a, the three adjacent longitudinal elements 16(1), 16(2), 16(3) have a mutual distance in the tangential direction as defined by a slot between them resulting from the cutting process by means of which they were made out of a tube. The situation of FIG. 19a may thus be the situation directly after the manufacturing is finished. However, e.g., longitudinal element 16(2) has a wider width some longitudinal distance away from the cross section shown in FIG. 19a. If then this wider portion is shifted to the cross section location of FIG. 19a, this wider portion will abut both adjacent longitudinal elements 16(1) and 16(3) at the cross section location shown in FIG. 19a and push against them in the tangential direction. As a result also a radial force outward from central axis 1900 is generated pushing longitudinal elements 16(1), 16(2), 16(3) in the radial direction towards outer cylindrical element 4, as shown in FIG. 19b. This may compensate or eliminate radial play between adjacent cylindrical elements.

It is observed that the embodiment shown in FIGS. 19a and 19b may be similar or identical to the one shown in FIGS. 17a-17f, 18a, 18b, 18c and 18d. However, other embodiments are possible. For instance, longitudinal element 16(1) and/or longitudinal element 16(3) may be substituted by a another portion of cylindrical element 3 like a floating spacer also resulting from the manufacturing process, e.g., laser cutting in a tube, or a spacer attached to rigid end party 10 or 15 (cf. FIG. 2) and extending longitudinally, at least partly, along longitudinal element 16(2).

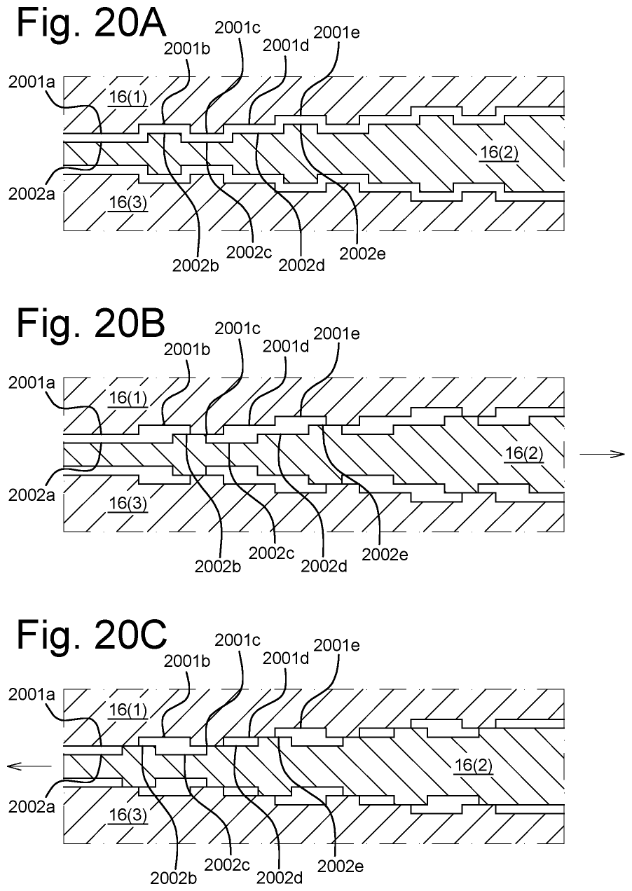

These play compensation methods can also be used on tapered longitudinal elements or on every other element like spacers or guiding elements used in steerable instruments. An example is shown in FIGS. 20a-20c.

In the embodiment of FIGS. 20a-20c, longitudinal element 16(1) has a longitudinal side facing a longitudinal side of longitudinal element 16(2). That longitudinal side of longitudinal element 16(1) is provided with a plurality of consecutive extending and indented portions 2001a, 2001b, 2001c, 2001d, 2001e, etc. Likewise, that longitudinal side of longitudinal element 16(2) is provided with a plurality of consecutive extending and indented portions 2002a, 2002b, 2002c, 2002d, 2002e, etc. the manufacturing process has been such that, in general, the shown portion of longitudinal element 16(2) tapers towards the left hand side, i.e., the width of longitudinal element 16(2) in the tangential direction of the instrument becomes less the more left in FIGS. 20a-20c. The most left hand side may be the distal end of the instrument, whereas the right hand side may be the proximal end, but that may also be the other way around.

FIG. 20a shows the situation directly after the manufacturing is finished, i.e., the manufactured state. Then, each extending/indented portion 2001a-2001e of longitudinal element 16(1) is located opposite a corresponding extending/indented portion of longitudinal element 16(2) at a distance as defined by the slot between them. These distances may, at that moment, be equal.

In the embodiment of FIGS. 20a-20c, a side portion of longitudinal element 16(1), 16(2) is called an extending portion if it has an indented portion at at least one longitudinal side where “indented” is relative to the extension of the extending portion. So, portion 2001a is an extending portion relative to portion 2001b, portion 2001c is an extending portion relative to portions 2001b and 2001d, and portion 2001d is an extending portion relative to portion 2001e but an indented portion relative to portion 2001c. Also, portion 2002b is an extending portion relative to portions 2002a and 2002c, portion 2002d is an extending portion relative to portion 2002c but an indented portion relative to portion 2002e, and portion 2001e is an extending portion relative to portion 2001d.

The opposite longitudinal side of longitudinal element 16(2) may have a similar extending/indented structure. In the shown example of FIGS. 20a-20c, this structure at the opposite longitudinal side is mirror symmetric relative to the side with extending/indented portions 2002, . . . , 2002e, . . . , where mirror symmetric is seen relative to a central axis of longitudinal element 16(2). Longitudinal element 16(3) may also have such an extending/indented structure along its side facing longitudinal element 16(2), as shown.

If longitudinal element 16(2) is tapering towards the left hand side of the drawing, then, both longitudinal elements (16(1), 16(3) may be tapering in the opposite longitudinal direction, as shown.

Transition edge portions between adjacent extending and indented portions are shown to be perpendicular to the longitudinal direction. However, they may be angled as explained above with reference to FIGS. 17d-17f.

FIG. 20b shows a situation in which longitudinal element 16(2) is shifted sideways to the right hand side relative to both longitudinal elements 16(1), 16(3). This relative shift may be to one of them only. As shown, all extending portions 2002b, 2002e will then abut an opposing extending/indented portion of the opposite longitudinal side of longitudinal element 16(1). However, the extending/indented portion structure of both opposing longitudinal side of longitudinal elements 16(1), 16(2) may be designed such that at least one extending/indented portion 2002a, . . . , 2002e, . . . then abuts an extending/indented portion 2001a, . . . , 2001e, . . . . In this situation, tangential play is removed at all longitudinal locations where an extending/indented portion 2002a, 2002e, . . . abuts an extending/indented portion 2001a, . . . , 2001e, . . . . This situation is already obtained when the relative shift between longitudinal element 16(2) and longitudinal element 16(1) and/or 16(3) is as large as the width of the slot between two opposing tangential sides of an extending/indented portion 2002a, . . . , 2002e, . . . and an extending/indented portion 2001a, . . . , 2001e, . . . . As observed hereinbefore, this width may be very small, e.g., as small as between 0.01 and 2.00 mm, more typically for this application, between 0.015 and 0.04 mm. in use, the instrument may be inserted in a curved channel which may automatically force adjacent longitudinal elements to shift a little bit relative to one another and, then, causing a situation as shown in FIG. 20b.

In an embodiment, after longitudinal element 16(2) has been shifted to the right relative to longitudinal element 16(1) and/or 16(3), as shown, still some play may be left in dependence on the tangential heights of the respective extending/indented portions. So, tangential play may be reduced or entirely eliminated.

FIG. 20c shows a situation in which longitudinal element 16(2) is shifted sideways to the left hand side relative to both longitudinal elements 16(1), 16(3). This relative shift may be to one of them only. As shown, all extending portions 2002b, 2002e will then abut an opposing extending/indented portion of the opposite longitudinal side of longitudinal element 16(1). However, the extending/indented portion structure of both opposing longitudinal side of longitudinal elements 16(1), 16(2) may be designed such that at least one extending/indented portion 2002a, . . . , 2002e, . . . then abuts an extending/indented portion 2001a, . . . , 2001e, . . . . In this situation, tangential play is removed at all longitudinal locations where an extending/indented portion 2002a, . . . , 2002e, . . . abuts an extending/indented portion 2001a, . . . , 2001e, . . . . This situation is already obtained when the relative shift between longitudinal element 16(2) and longitudinal element 16(1) and/or 16(3) is as large as the width of the slot between two opposing tangential sides of an extending/indented portion 2002a, . . . , 2002e, . . . and an extending/indented portion 2001a, . . . , 2001e, . . . . As observed hereinbefore, this width may be very small, e.g., as small as between 0.01 and 2.00 mm, more typically for this application, between 0.015 and 0.04 mm. in use, the instrument may be inserted in a curved channel which may automatically force adjacent longitudinal elements to shift a little bit relative to one another and, then, causing a situation as shown in FIG. 20c.

In an embodiment, after longitudinal element 16(2) has been shifted to the left relative to longitudinal element 16(1) and/or 16(3), as shown, still some play may be left in dependence on the tangential heights of the respective extending/indented portions. So, tangential play may be reduced or entirely eliminated.

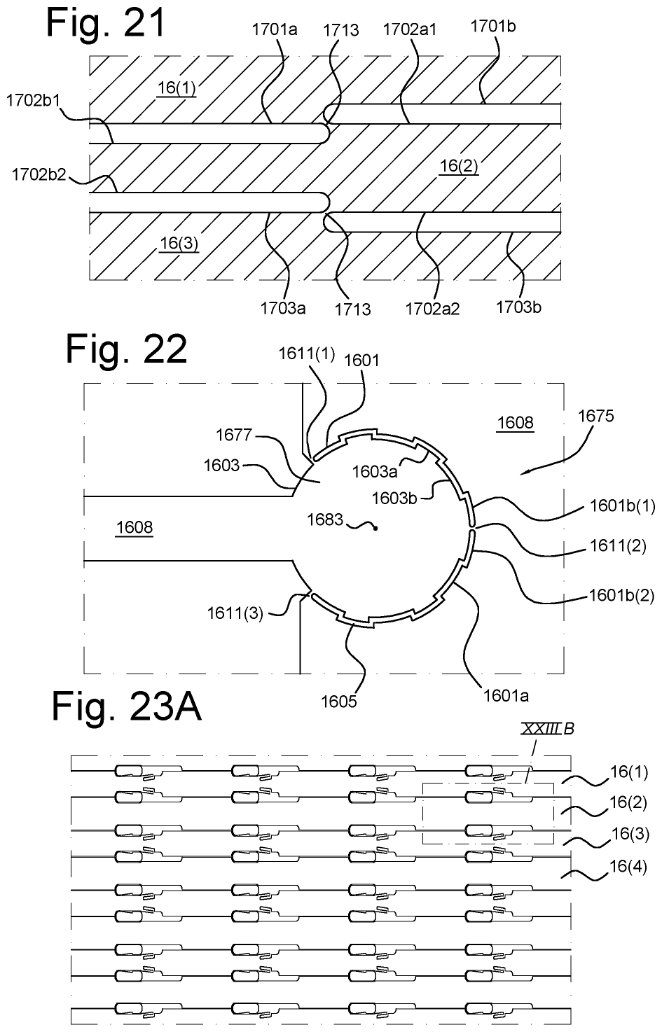

FIG. 21 shows three adjacent longitudinal elements 16(1), 16(2), 16(3) in the same arrangement as in FIG. 17a. FIG. 21 shows them in the arrangement directly after the manufacturing is finished, i.e., making the slots 1705 in a cylindrical element with e.g. a laser cutting process. However, in the embodiment of FIG. 21, one or more of the adjacent longitudinal elements 16(1), 16(2), 16(3) are still attached to one another by means of one or more fracture elements 1713. As explained above, such fracture elements keep the different longitudinal elements together during further assembly of the instrument, e.g., when one cylindrical element is inserted into another one. In use, i.e., when a predetermined minimum force is applied to shift longitudinal element 16(2) relative to longitudinal element 16(1) and/or 16(3) these fracture elements 1713 will fracture and play no role anymore. In the example of FIG. 21, a fracture element 1713 is provided between opposite transition edge portions of opposite extending/indented portions 1701a/1701b-1702b1/1702a1, 1703a/1703b-1702b2/1702a2. Of course, there can be more such fracture elements at those locations and/or at other locations between opposite longitudinal sides of adjacent longitudinal elements 16(1), 16(2), 16(3).

FIG. 22 shows a portion of a hinge like the one shown in FIGS. 16a-16e. FIG. 22 shows the hinge segments 1608 in the arrangement directly after the manufacturing is finished, i.e., making the slots 1605 in a cylindrical element with e.g. a laser cutting process. However, in the embodiment of FIG. 22, the hinge segments 1608 are still attached to one another by means of one or more fracture elements 1611(k) (k=1, 2, . . . , K). As explained above, such fracture elements keep the different hinge segments 1608 together during further assembly of the instrument, e.g., when one cylindrical element is inserted into another one. In use, i.e., when a predetermined minimum force is applied to rotate hinge segments 1608 relative to one another these fracture elements 1611(k) will fracture and play no role anymore. In the example of FIG. 22, three such fracture elements 1611(k) are provided. Of course, there can be more such fracture elements.

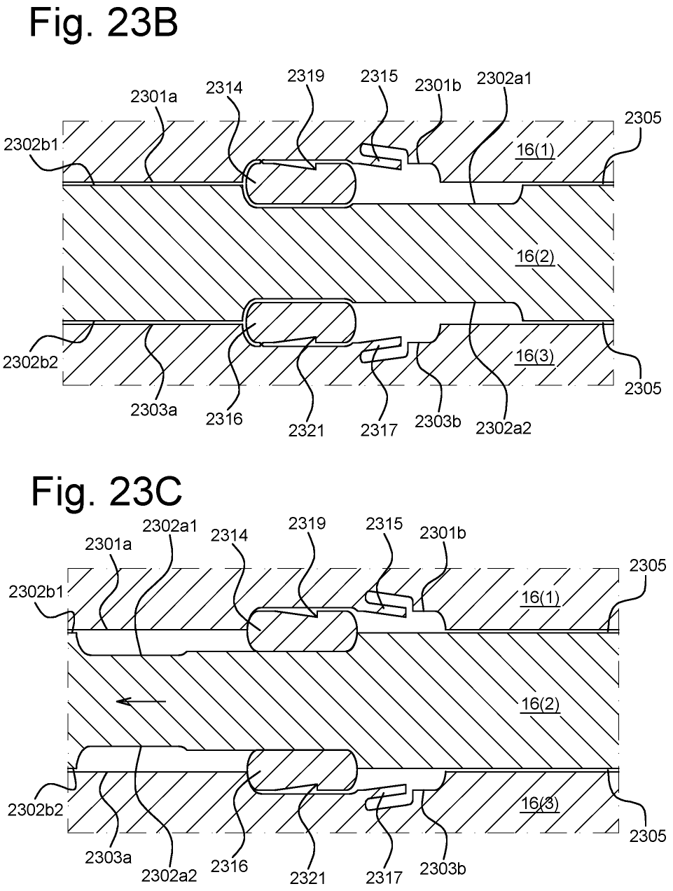

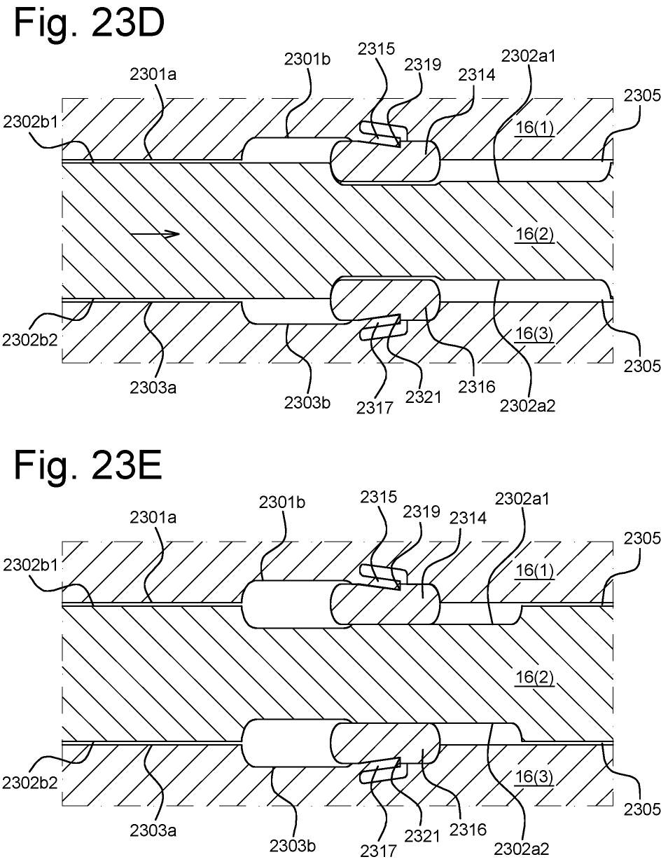

FIGS. 23a-23e show an embodiment in which a spacer arranged between two adjacent longitudinal elements 16(1), 16(2), 16(3), . . . but, in use, not attached to either one of the adjacent longitudinal elements 16(1), 16(2), 16(3), . . . operates as a tangential play reducing or eliminating element. Alternatively, either longitudinal element 16(1) or 16(3) or both are substituted by other parts of the cylindrical element not operating as longitudinal elements. FIGS. 23a and 23b show the embodiment in its manufactured state.

FIGS. 23b-23e are enlarged views of a portion of adjacent longitudinal elements 16(1), 16(2), 16(3) shown in FIG. 23a.

As shown in detail in FIG. 23b, longitudinal element 16(1) has a longitudinal side adjacent to a longitudinal side of longitudinal element 16(2). They are separated from each other by a slot 2305 resulting from a (laser) cutting process in a cylindrical element. The longitudinal side of longitudinal element 16(1) has extending portions 2301a. Between two such extending portions 2301a, the longitudinal side of longitudinal element 16(1) has an indented portion 2301b. In indented portion 2301b the longitudinal side of longitudinal element 16(1) is provided with a flexible portion 2315 extending towards the longitudinal side of longitudinal element 16(2). Flexible portion 2315 operates as a spring.

The longitudinal side of longitudinal element 16(2) has extending portions 2302b1. Between two such extending portions 2302b1, the longitudinal side of longitudinal element 16(2) has an indented portion 2302a1. In the shown embodiment, indented portion 2302a1 has a larger longitudinal length than the longitudinal length of indented portion 2301b. However, that may be different.

Indented portions 2301b and 2302a1 together form an open space between the two adjacent longitudinal elements 16(1), 16(2). A spacer 2314 is located inside that open space. Spacer 2314 is provided with a indented portion 2319 arranged at a side facing longitudinal element 16(1). Directly after the manufacturing process is finished spacer 2314 is, preferably, still attached to at least one of the opposite longitudinal sides of longitudinal elements 16(1), 16(2) by means of one or more fracture elements, in order to prevent spacer 2314 from falling apart from the rest of the cylindrical element. At least one of indented portion 2301b or 2302a1 may be slightly further indented at a location adjacent spacer 2314 such that spacer 2314 cannot move freely in the longitudinal direction towards flexible portion 2315. In the shown embodiment, flexible portion 2315 extends towards adjacent longitudinal element 16(2) and also away from spacer 2314 such that it can be flexibly moved in the tangential direction of the cylindrical element.

As also shown in detail in FIG. 23b, longitudinal element 16(3) has a longitudinal side adjacent to another longitudinal side of longitudinal element 16(2). They are separated from each other by a slot 2305 resulting from a (laser) cutting process in a cylindrical element. The longitudinal side of longitudinal element 16(3) has extending portions 2303a. Between two such extending portions 2303a, the longitudinal side of longitudinal element 16(3) has an indented portion 2303b. In indented portion 2303b the longitudinal side of longitudinal element 16(3) is provided with a flexible portion 2317 extending towards the other longitudinal side of longitudinal element 16(2). Flexible portion 2317 operates as a spring.

The other longitudinal side of longitudinal element 16(2) has extending portions 2302b2. Between two such extending portions 2302b2, the longitudinal side of longitudinal element 16(2) has an indented portion 2302a2. In the shown embodiment, indented portion 2302a2 has a larger longitudinal length than the longitudinal length of indented portion 2303b. However, that may be different.

Indented portions 2303b and 2302a2 together form an open space between the two adjacent longitudinal elements 16(3), 16(2). A spacer 2316 is located inside that open space. Spacer 2316 is provided with a indented portion 2321 arranged at a side facing longitudinal element 16(3). Directly after the manufacturing process is finished spacer 2316 is, preferably, still attached to at least one of the opposite longitudinal sides of longitudinal elements 16(3), 16(2) by means of one or more fracture elements, in order to prevent spacer 2316 from falling apart from the rest of the cylindrical element. At least one of indented portion 2303b or 2302a2 may be slightly further indented at a location adjacent spacer 2316 such that spacer 2316 cannot move freely in the longitudinal direction towards flexible portion 2317. In the shown embodiment, flexible portion 2317 extends towards adjacent longitudinal element 16(2) and also away from spacer 2316 such that it can be flexibly moved in the tangential direction of the cylindrical element.

FIGS. 23c-23e explain how spacers 2314 and 2316, respectively, can be moved in the longitudinal direction such that flexible portions 2315 and 2317, respectively, will be moved into indented portions 2319 and 2321, respectively, such that spacers 2314 and 2316 will be locked relative to longitudinal element 16(1) and 16(3), respectively, and also operate as tangential play reducing or eliminating elements.

At a first actuation, longitudinal element 16(2) is moved relative to adjacent longitudinal elements 16(1), 16(3). In the shown example, indented portions 2302a1 and 2302a2 are dimensioned such that longitudinal element 16(2) can be pushed to the left hand side without causing any change in the cut pattern. Longitudinal element 16(2) is moved to such an extent relative to longitudinal elements 16(1), 16(3) that any fracture element attaching spacer 2314 and 2316, respectively, to at least one of adjacent longitudinal elements 16(1) or 16(2), or adjacent longitudinal elements 16(2) and 16(3), is fractured such that spacer 2314, 2316 become freely floating inside the respective open spaces between the adjacent longitudinal elements in which they are located. This is shown in FIG. 23c.

Then longitudinal element 16(2) is moved towards the right relative to adjacent longitudinal elements 16(1) and 16(3) and spacers 2314 and 2316, respectively, are pulled by transition sides between indented portion 2302a1 and extending portion 2302b1 and between indented portion 2302a2 and extending portion 2302b2, respectively, of longitudinal element 16(2) in the right direction too. When spacers 2314 and 2316, respectively, are moving to the right they will be pushed against flexible portions 2315 and 2317, respectively, such that flexible portions 2315 and 2317, respectively, will both be bent in opposite tangential directions, i.e., towards longitudinal element 16(1) and 16(3), respectively. Moving forward in the right direction, at a certain moment in time, flexible portions 2315 and 2317, respectively, will automatically click inside indented portions 2319 and 2321, respectively, such that spacers 2314 and 2316 are locked in place. As shown in FIG. 23d, further movement of spacers 2314 and 2316, respectively, towards the right may be prevented by transition edge portions of indented portions 2301b and 2303b, respectively, towards extending portions 2301a and 2303a, respectively. In FIG. 23d longitudinal element 16(2) is shifted to its most right hand position relative to longitudinal elements 16(1), 16(3).

Again, these transition edge portions may be straight and oriented tangentially, but may also be angled relative to the tangential direction. They may also be curved as shown in FIGS. 23a-23e.

It is observed that the order of movements as shown in FIGS. 23c and 23d may be caused by bending the cylindrical element in which longitudinal elements 16(1), 16(2), 16(3) are located because that results in a relative longitudinal movement of the longitudinal elements 16(1), 16(2), 16(3). However, the same bending action results in a reversed order of relative movements of longitudinal elements at the opposite side of the cylindrical element, i.e., at the opposite side the order of FIGS. 23c and 23d is reversed.

Whereas FIG. 23a shows all spacers 2314, 2316 being located at the left hand side of flexible portions 2315, 2317, some of these relative orientations may be the other way around.

FIG. 23e shows the final situation in which longitudinal elements 16(1), 16(2), 16(3) are shifted back relative to one another as in the situation of FIG. 23a, i.e., the situation directly after the cutting process. Both spacers 2314 and 2316, respectively, are now locked in place such that they cannot move in the longitudinal direction relative to longitudinal element 16(1) and 16(3), respectively. In this final situation, flexible portions 2315 and 2317, respectively, push against spacers 2314 and 2316, respectively, such that they are both pushed towards longitudinal element 16(2). Consequently, at the locations of these spacers 2314, 2316, all tangential play is eliminated. The force with which spacers 2314, 2316 are pushed against longitudinal element 16(2) depends on the tangential width of spacers 2314, 2316 and the spring force of flexible portions 2315, 2317 in the final stage of FIG. 23e. This force may be zero in the final stage of FIG. 23e. However, if some friction is required, flexible portion 2315, 2317 may still be bent in the situation of FIG. 23e and exert some force equal to or larger than zero.

Now longitudinal elements 16(1), 16(2), 16(3) can move back and forth relative to one another, while the play reduction or elimination is permanent also in the longitudinal elements neutral positions.

The spacers 2314, 2316 can be circular, rectangular or any other desired shape. They can be as short as 0.1 mm to 5 mm. However, in theory, they can have any length up to the full instrument length. One can also think of other shape elements that are fixed in an as cut position and are relocated in the instrument in a position that eliminates tangential play, radial play or both. It is observed that the arrangements shown in FIGS. 23a-23e are not restricted to longitudinal elements but can equally be implemented between hinge segments in hinges which are configured to rotate relative to one another in use.

In FIGS. 23a-23e a sliding oval spacer is drawn, but one can also think of a shape that is rotated or bent when the longitudinal steering wire is actuated and which will reside in that position after actuation and which will reduce tangential play in that position.

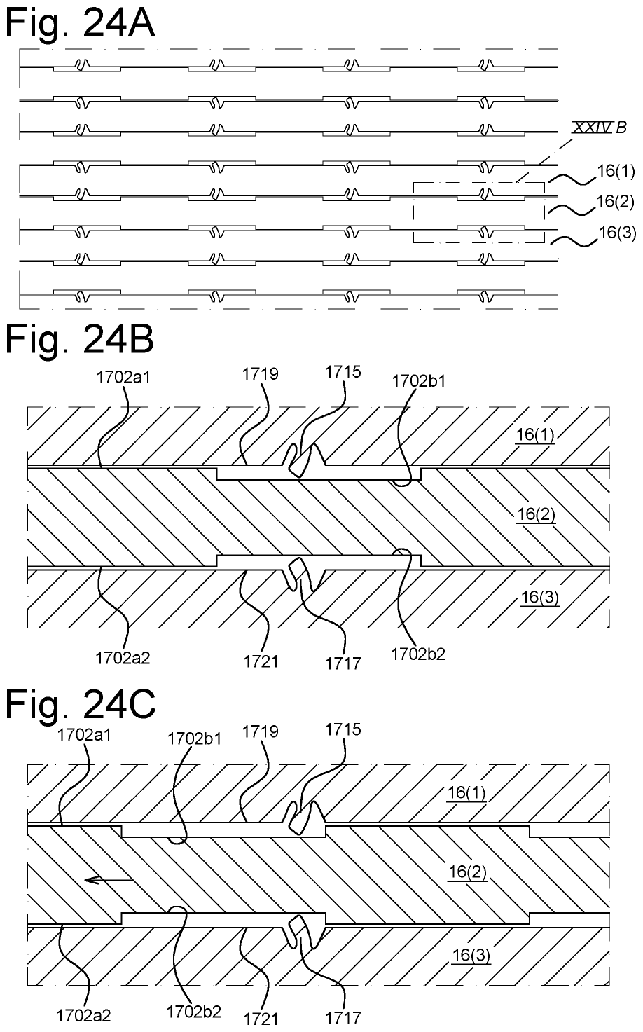

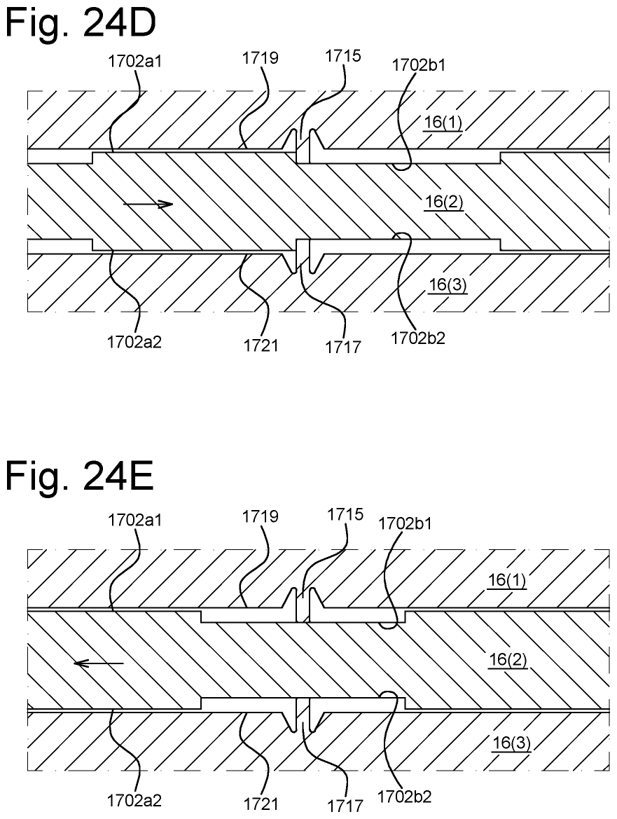

For example a bending element is shown in FIGS. 24a-24e. I.e., these figures show three adjacent longitudinal elements 16(1), 16(2), 16(3). However, again longitudinal element 16(1) and/or longitudinal element 16(3) can be substituted by another portion of the cylindrical element not having the function of a longitudinal element. FIGS. 24a and 24b show the embodiment in its manufactured state.

Longitudinal element 16(1) has a longitudinal side 1719 facing a longitudinal side of longitudinal element 16(2). That longitudinal side of longitudinal element 16(2) has one or more extending portions 1702a1 and one or more indented portions 1702b1. Longitudinal side 1719 of longitudinal element 16(1) is provided with a bending element 1715 which, directly after finishing the cutting process in the cylindrical element, is oriented at an angle relative to the tangential direction of the instrument larger than 0 degrees but smaller than 90 degrees. Moreover, in its original state, bending element 1715 is slightly curved.

Longitudinal element 16(3) has a longitudinal side 1721 facing another longitudinal side of longitudinal element 16(2). That other longitudinal side of longitudinal element 16(2) has one or more extending portions 1702a2 and one or more indented portions 1702b2. Longitudinal side 1721 of longitudinal element 16(3) is provided with a bending element 1717 which, directly after finishing the cutting process in the cylindrical element, is oriented at an angle relative to the tangential direction of the instrument larger than 0 degrees but smaller than 90 degrees. Moreover, in its original state, bending element 1717 is slightly curved. In the arrangement shown in FIG. 24b the orientations of bending elements 1715 and 1717 are the same, but they may have opposite orientations.

FIG. 24c shows a situation in which longitudinal element 16(2) is longitudinally shifted sideways relative to its adjacent longitudinal elements 16(10 and 16(3) in the left hand direction. Again this may be caused by a bending action of the cylindrical element in which the longitudinal elements 16(1), 16(2), 16(3) are located. In FIG. 24d, longitudinal element 16(2) is longitudinally shifted sideways relative to its adjacent longitudinal elements 16(10 and 16(3) in the right hand direction such that transition edge portions between intended portions 1702b1, 1702b2 and extending portions 1702a1, 1702a2 also move the tip of bending portions 1715 and 1717 towards the right. After this action, both bending elements 1715 and 1717 have a more straight orientation and their tips now abut indented portions 1702b1 and 1702b2, respectively.

Bending portions 1715 and 1717 are designed such that when longitudinal element 16(2) is consecutively moved relative to longitudinal elements 16(1), 16(3) to the left again, as shown in FIG. 24e, they keep a more straightened orientation and keep abutting indented portions 1702b1 and 1702b2, respectively.

In the situation of FIG. 24e, longitudinal elements 16(1), 16(2), 16(3) can freely move relative to one another in the longitudinal direction wherein bending elements 1715 and 1717 operate as play eliminating elements. Alternatively, in the situations of FIGS. 24d and 24e bending elements 1715 and 1717 remain at a certain predetermined distance from longitudinal element 16(2) such that play is reduced but not entirely eliminated.

It is observed that by the actions shown in FIGS. 24c, 24d and 24e, some of the bending elements 1715, 1717 may also be caused to bend slightly in a radial direction of the cylindrical element such that they also operate as radial spacers and reducing and/or eliminating radial play in the instrument because they will touch another cylindrical element inside or outside their own cylindrical element.

It is observed that the arrangements shown in FIGS. 24a-24e are not restricted to longitudinal elements but can equally be implemented between hinge segments in hinges which are configured to rotate relative to one another in use.

A steerable instrument made by creating parts integrally from the wall of a cylindrical element has limitations. One can only create 2 dimensional geometries with a certain thickness. One can of course create 3 dimensional parts from the wall of a tube by (locally) changing the thickness of the wall by for example laser ablation, etching or chipping techniques, but in practice those might be difficult processes. Therefore, all the above-mentioned techniques to manage play between parts are based on achieving control over play by applying the invention in one cylindrical element wall which can have a uniform thickness. An alternative approach to manage play between parts in an instrument that is made from cylindrical elements is to use more than one cylindrical element to set play between parts at the desired magnitude. For example, if one would make longitudinal elements in two layers one can eliminate tangential play by attaching the longitudinal element guiding in one cylindrical element to a longitudinal element guiding in the next cylindrical element and attach the composite longitudinal element guiding to an inner or outer cylindrical element. If one moves the longitudinal elements in one cylindrical element to a position with preferred play between the longitudinal element and the longitudinal element guiding and one moves the longitudinal element in the other cylindrical element to an opposed position with preferred play between that longitudinal element and its longitudinal element guiding and if one attaches this longitudinal element to the longitudinal element in the next cylindrical element, tangential play between the composite longitudinal element and the composite longitudinal element guiding is eliminated or set to the preferred magnitude.

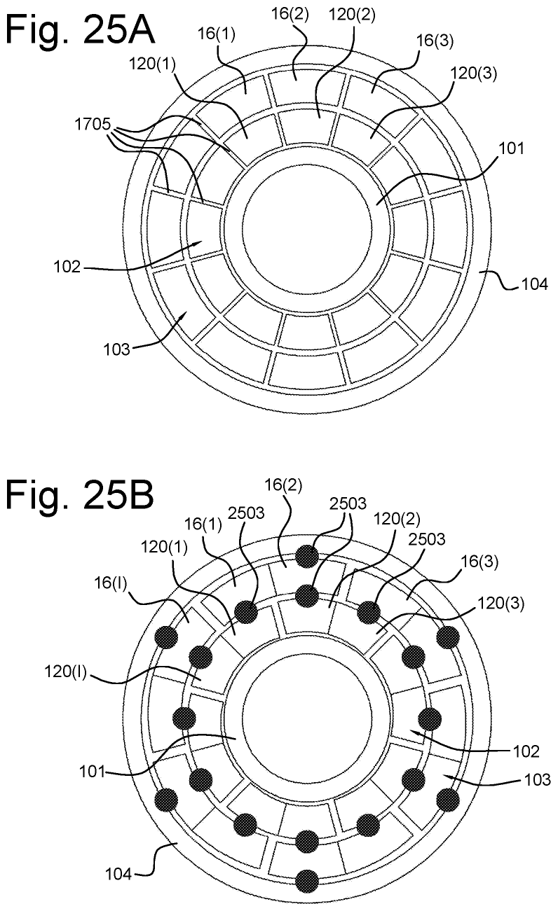

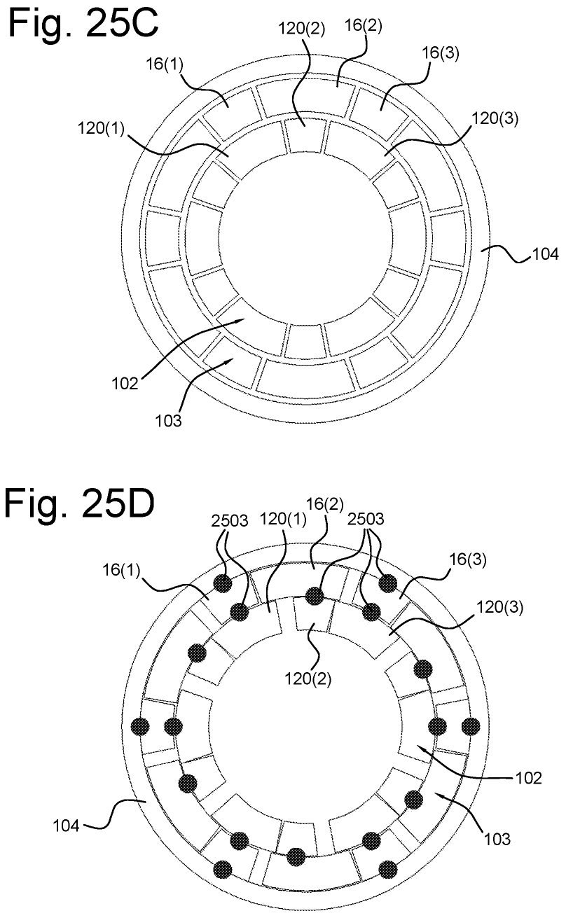

This will be explained in further detail with reference to FIGS. 25a-25d. FIG. 25a shows a cross section through an invasive instrument having four cylindrical elements: inner cylindrical element 101, first intermediate cylindrical element 102, second intermediate cylindrical element 103, and outer cylindrical element 104. They are surrounding one another in this order. Second intermediate cylindrical element 103 comprises adjacent longitudinal elements 16(1), 16(2), 16(3), which are also shown in several other figures above. FIG. 25a shows how they result from (laser) cutting from cylindrical element 103, i.e., in the tangential direction they are curved because of the tangential curvature of the cylindrical element 103 from which they originate. Moreover, FIG. 25a shows how longitudinal elements 16(1), 16(2), 16(3), . . . , 16(l) are separated from one another by slots 1705 resulting from the cutting process. First intermediate cylindrical element 102 also comprises a plurality of longitudinal elements 120(1), 120(2), 120(3), . . . , 120(l) which, in FIG. 25a are also separated from one another by slots 1705 resulting from the cutting process. Longitudinal elements 120(1), 120(2), . . . 120(l), respectively, are located radially inside longitudinal elements 16(1), 16(2), . . . 16(l), respectively.

FIG. 25b shows how adjacent longitudinal elements 16(1), 16(2) are tangentially moved towards another such that they are at a first predetermined tangential distance from one another, which may be 0 mm (physical contact). Moreover, adjacent longitudinal elements 120(1) and 120(2), respectively, are moved tangentially away from one another such that they are at a second predetermined tangential distance from their other respective adjacent longitudinal elements 16(l) and 120(l), respectively, which distance may, again, be 0 mm (physical contact). When these longitudinal elements 16(1), 16(2), 16(l), 120(1), 120(2), 120(l) are in this status, longitudinal elements 16(2), 16(l) are attached to outer cylindrical element 104 by means of an attachment 2503, and longitudinal elements 16(1), 16(2), and 16(l), respectively, are attached to longitudinal elements 120(1), 120(2), and 120(l), respectively, by means of attachments 2503.

The attachments 2503 can be implemented by, for example, (laser) welding, brazing, bonding, gluing, or by, for example, bending folding tabs in one cylindrical element/longitudinal element into recesses in the adjacent other cylindrical element/longitudinal element. Then, longitudinal elements 16(2), 16(l), respectively, show no or only minimal tangential play relative to both outer cylindrical element 104 and longitudinal element 120(2), 120(l), respectively. Moreover, longitudinal element 16(1) shows no or only minimal tangential play relative to longitudinal element 120(1). In the embodiment of FIG. 25b, longitudinal element 16(1) shows no or only minimal tangential play relative to longitudinal element 16(2) while longitudinal element 16(1) is still able to move longitudinally relative to longitudinal element 16(2). At the same time, longitudinal element 120(1) to which longitudinal element 16(1) is attached shows no or only minimal tangential play relative to longitudinal element 120(l) while longitudinal element 120(1) is still able to move longitudinally relative to longitudinal element 120(l).

It is observed that, in the embodiment of FIGS. 25a, 25b, one or more of the longitudinal elements 16(2), 120(2), 16(l), 120(l) can be replaced by other portions cut from cylindrical elements 102, 103, such as spacers. Moreover, longitudinal element 16(2) need not be attached to outer cylindrical element 104 by means of attachment 2503, such that also longitudinal elements 16(2), 120(2) can still move in the longitudinal direction. In another embodiment, attachment 2503 between longitudinal elements 16(2) and 120(2) is absent.

To summarize the idea of FIG. 25b, longitudinal element 16(1) of second intermediate cylindrical element 103 is at the first tangential distance from an adjacent portion 16(2) of the second intermediate cylindrical element 103 which adjacent portion 16(2) is attached to outer cylindrical element 104. The first tangential distance may be 0 mm. Moreover, longitudinal element 120(1) of first intermediate cylindrical element 102 is at the second tangential distance from an adjacent portion 120(l) of the first intermediate cylindrical element 102 which adjacent portion 120(l) is also attached to outer cylindrical element 104—here, via longitudinal element 16(l). The second tangential distance may also be 0 mm.

The embodiment of FIGS. 25c, 25d is a variant to the one of FIGS. 25a, 25b. The difference is that longitudinal element 16(2) has such a larger width than longitudinal element 120(2) that longitudinal element 16(2) is not only radially adjacent to longitudinal element 120(2) but also partly radially adjacent to at least one of longitudinal element 120(1) or 120(3). Moreover, now, longitudinal element 16(2) is not attached to outer cylindrical element 104 but longitudinal elements 16(1) and 16(3) are attached to outer cylindrical element 104. In this way, longitudinal elements 16(2) and 120(2), which are attached to one another, are capable of moving longitudinally without any (or hardly any) tangential play between, at one tangential side, a set of longitudinal elements 16(1), 120(1) attached to one another and to outer cylindrical element 104, and, at the other tangential side, a set elements 16(3), 120(3) attached to one another and to outer cylindrical element 104, as well. Moreover, in the situation of FIG. 25d, longitudinal element 16(2) is still radially adjacent to at least one of longitudinal elements 120(1) and 120(3), such that it is radially locked. Therefore, in the embodiment of FIGS. 250, 25d inner cylindrical element 101 can be left out. Another advantage is that also radial play is set at zero.

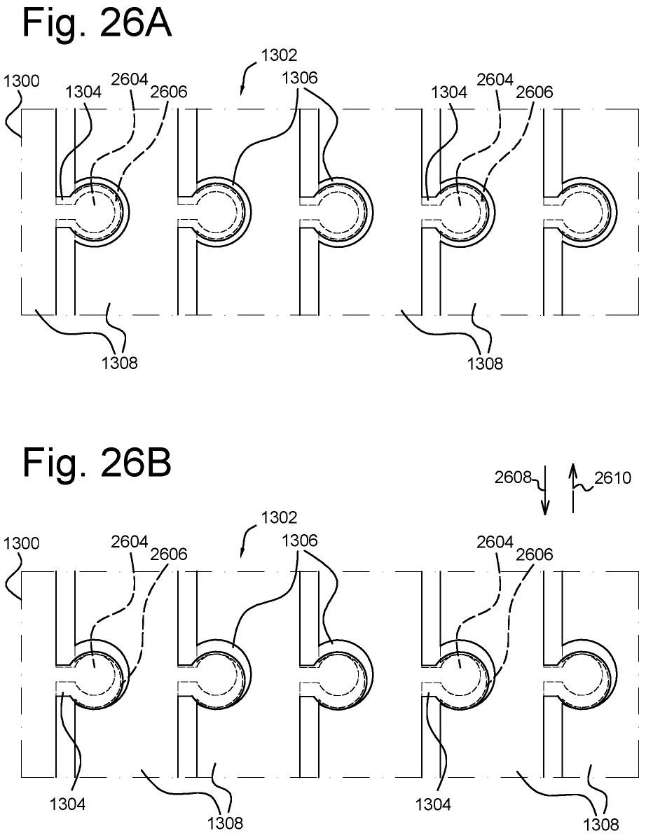

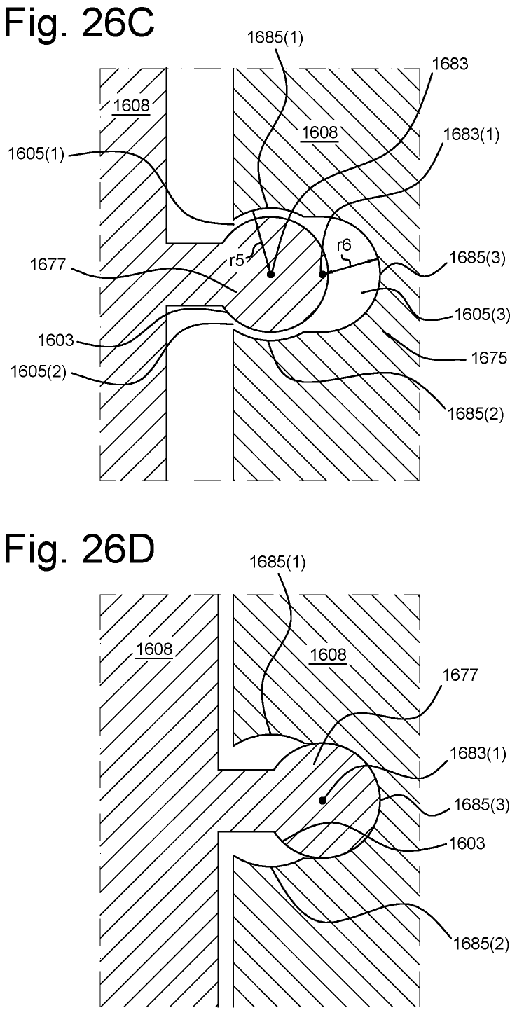

The same method can be applied for hinges that are used in for example the flexible zones in an instrument. One can now preset tangential and longitudinal play to a preferred value in hinges too. An example is shown in FIGS. 26a, 26b.

The example of FIGS. 26a, 26b comprises two adjacent cylindrical elements, one surrounding the other. The outside one is drawn in solid lines, the inner one in dashed lines. The schematic drawings of FIGS. 26a, 26b show hinge 1302 with the same components as shown in FIGS. 13a-13c, but the implementation may be different. The inside cylindrical element has an inside hinge at the same longitudinal location as hinge 1302. This inside hinge may have the same structure as hinge 1302. At least, the inside hinge has inside convex portions 2604 and inside concave portions 2606. Each inside convex portion 2604 is located radially inside a convex portion 1304 and each inside concave portion 2606 is located radially inside a concave portion 1306. Moreover, each inside convex portion 2604 is longitudinally arranged inside an inside concave portion 2606 and separated from it by means of a slot resulting from the (laser) cutting process.

As indicated with arrow 2608 in FIG. 26b, convex portion 1304 is shifted relative to concave portion 1306 in a first tangential direction 2608 such that their mutual tangential distance in the first tangential direction 2608 is reduced or even eliminated. Moreover, inside concave portion 2604 is shifted relative to inside concave portion 2606 in a second tangential direction 2610 such that their mutual tangential distance in the second tangential direction 2608 is reduced or even eliminated. The first and second tangential directions are opposite one another.

Once these shifts have been executed, convex portion 1304 and its underlying inside convex portion 2606 are attached, e.g., by (laser) welding, brazing, bonding, gluing, or by, for example, bending folding tabs from one of them into a suitable hole in the other one. Then, convex portion 1304 and inside convex portion 2604 are tangentially fixed to one another while, moreover, tangential play of the attached convex portion 1304 and inside convex portion 2604 is reduced or even eliminated in a similar way as explained with reference to longitudinal elements in FIGS. 25a-25d.

Here, a convex portion 1304 of a first hinge segment 1308 has reduced or even zero play in the first tangential direction relative to an adjacent concave portion 1306 of an adjacent hinge segment 1308. Moreover, an inside convex portion 2604 of a first inside hinge segment has reduced or even zero play in the second tangential direction relative to an adjacent inside concave portion 1306 of an adjacent inside hinge segment. It will be evident that reduced tangential play is already obtained by performing at least one of the first or second relative shifts as explained above before attaching convex portion 1304 to inside convex portion 2604.

It is observed that a similar method can be used to reduce or even eliminate longitudinal play in hinges by substituting the term “tangential” for “longitudinal” in the above explanation.

A variant of the embodiment of FIGS. 26a, 26b is shown in FIGS. 26c, 26d.

FIGS. 26a and 26b show a special embodiment by means of which play in a hinge can be removed totally in a finished instrument. FIG. 26a shows a portion of a hinge in an unfinished state of the instrument and FIG. 26d in a finished state. Like the other figures relating to hinges, these are very schematic drawings. They show two adjacent hinge segments 1608 with a convex portion 1677 and a concave portion 1675.

FIG. 26c shows both hinge segments 1608 after the cutting process in the cylindrical element is finished. Because of the cutting process, convex portion 1677 and concave portion 1675 are separated from one another by a slot 1605(1), 1605(2), 1605(3) and show play relative to one another. In the embodiment of FIG. 26c, the slot has a first slot portion 1605(1) and a second slot portion 1605(2) with a smaller width and a third slot portion 1605(3) with a much larger width. Slots 1605(1) and 1605(2) extend at tangential sides, i.e. the vertical direction in the drawing, of convex portion 1677. The third slot portion 1605(3) is located between convex portion 1677 and concave portion 1675 in the longitudinal direction, i.e. the horizontal direction of the drawing, of the instrument. The width of slots 1605(1) and 1605(2) may the minimum width obtainable by the used cutting process.

In the embodiment of FIGS. 26c, 26d convex portion 1677 is at least partly circular about center point 1683. Slots 1605(1) and 1605(2) extend along these at least partly circular portions of convex portion 1677, which have a radius r5. In the embodiment, concave portion 1675 has a first concave edge 1685(1), a second concave edge 1685(2) and a third concave edge 1685(3). First concave edge 1685(1) is a sidewall of first slot portion 1605(1), second concave edge 1685(2) is a sidewall of second slot portion 1605(2), and third concave edge 1685(3) is a sidewall of third slot portion 1605(3). Third concave edge portion 1685(3) is at least partly circular about a center point 1683(1) with a radius r6. In the shown embodiment, the following equation holds: 0=<r6−r5<width of slot 1605(1).

After the arrangement of FIG. 26c has been made, convex portion 1677 is moved inside third slot 1605(3) such that center point 1683 coincides with center point 1683(1). After having done so, convex portion 1677 and concave portion 1675 are fixed relative to one another in the longitudinal direction such that they can still rotate relative to one another about center point 1683(1). Fixing may be done by providing longitudinal elements located in a cylindrical element inside or outside the cylindrical element in which the hinge is located with a pretension. Alternatively, the circle arc along which third edge portion 1685(3) extends may be larger than 180 degrees such that some force is required to move convex portion 1677 inside concave portion 1675 but once inside it remains inside. Stated differently, together they form a circular snap-fit connection. In the situation of FIG. 26d, play between convex portion 1677 and concave portion 1675 is reduced to r6-r5 which may be 0. This may be implemented both in the tangential and longitudinal direction of the instrument.

Another method for compensation of longitudinal or radial play is that one can cut a spiral in one cylindrical element. This spiral can be used as a longitudinal spring element to for example eliminate longitudinal play in hinges in the same cylindrical element or in a cylindrical element outside or inside this cylindrical element. A spiral, coil like, structure can also be used to compensate radial play. For example, if an inner cylindrical element has a coil like structure and the cylindrical element on top contains for example longitudinal elements, one can push the longitudinal elements radially outwards by rotating one end of the coil, as to ‘unwind’ the coil which increases its diameter, and fix the rotated end in the position where the desired amount of radial play is achieved between the longitudinal elements and the outer cylindrical element.

The following summarizing statements can be made.

The device with reduced play has two different states. The first state is called the manufactured state (alternatively, one may call it the “resting state”) which is the one obtained directly after manufacturing and the second state is one of reduced play in which the distance between two opposing extending portions is smaller than in the manufactured state. The reduced-play status coincides with an operation mode of the steerable instrument in which two opposing extending portions are sideways movable relative to one another (e.g. two portions of a hinge or two adjacent longitudinal elements like steering wires) along a predetermined maximum mutual displacement limit. In this reduced-play state, the two opposing extending portions slide along one another. There may be a third state between the manufactured state and the reduced-play state in which the distance between two opposing extending portions is larger than in the reduced-play state but smaller than in the manufactured state.

The movable element and the first further element may be opposite portions of a hinge and the operating of the cylindrical instrument then causes a deflection between the opposite portions of the hinge, wherein the predetermined maximum operating limit is a maximum deflection angle between the opposite portions of the hinge.

Then, the maximum deflection angle may have a value in a range of at least one of −2 to −45 degrees and +2 to +45 degrees.

The movable element may be a first longitudinal element extending in the longitudinal direction of the tube and the operating of the cylindrical instrument then causes a mutual longitudinal displacement between the longitudinal element and the first further element and the predetermined maximum operating limit is a maximum mutual longitudinal displacement.

The longitudinal element may be attached to a bendable portion of the tube at a distal end of the tube such as to transfer a longitudinal movement of the longitudinal element to a bending of the bendable portion.

Then, the maximum mutual longitudinal displacement may have a value in a range of at least one of −0.5 to −40 mm and +0.5 to +40 mm. This maximum operating limit of the movable element relative to the other element may depend on the longitudinal location in the instrument, i.e., for instance, a mutual longitudinal displacement between a steering wire and an adjacent element (e.g. another steering wire) may be much larger at the proximal end than at the distal end.

The predetermined maximum operating limit depends on the design specifications of the steerable invasive instrument, e.g., expressed in terms of maximum deflection angle of the steerable tip and maximum bending angle of adjacent hinge portions in a flexible body section of the instrument.

In the status of reduced play, one of the extending portions forms a sliding surface for the other, opposing extending surface. It is observed that in the shown embodiments both extending portions have a “smooth” surface (which may be curved, e.g. a circular form). However, one may be smooth whereas the other one may then be non-smooth, e.g., have a wavy pattern.

The wall thickness of cylindrical elements depend on their application. For medical applications the wall thickness may be in a range of 0.03-2.0 mm, preferably 0.03-1.0 mm, more preferably 0.05-0.5 mm, and most preferably 0.08-0.4 mm. The diameter of cylindrical elements depend on their application. For medical applications the diameter may be in a range of 0.5-20 mm, preferably 0.5-10 mm, more preferably 0.5-6 mm.

Longitudinal elements in one cylindrical element can be attached to longitudinal elements in adjacent cylindrical elements such that they are together operable to transfer a longitudinal motion from a longitudinal element at the proximal end of the instrument to a bendable portion of the instrument at the distal end of the instrument such that the bendable portion bends. This is explained in detail in WO 2017/213491 (cf. e.g. FIGS. 12, 13a and 13b in that PCT application) of the present applicant.

It will be clear to a person skilled in the art that the scope of the invention is not limited to the examples discussed in the foregoing, but that several amendments and modifications thereof are possible without deviating from the scope of the invention as defined in the attached claims. While the invention has been illustrated and described in detail in the figures and the description, such illustration and description are to be considered illustrative or exemplary only, and not restrictive. The present invention is not limited to the disclosed embodiments but comprises any combination of the disclosed embodiments that can come to an advantage.

Variations to the disclosed embodiments can be understood and effected by a person skilled in the art in practicing the claimed invention, from a study of the figures, the description and the attached claims. In the description and claims, the word “comprising” does not exclude other elements, and the indefinite article “a” or “an” does not exclude a plurality. In fact it is to be construed as meaning “at least one”. The mere fact that certain features are recited in mutually different dependent claims does not indicate that a combination of these features cannot be used to advantage. Any reference signs in the claims should not be construed as limiting the scope of the invention. Features of the above described embodiments and aspects can be combined unless their combining results in evident technical conflicts.

1. A cylindrical instrument comprising a tube extending in a longitudinal direction and having at least a movable element and at least a first further element, the cylindrical instrument having a first state and a second state, the first state being a manufactured state obtained directly after manufacturing and the second state being a reduce-play state coinciding with an operation mode, the movable element having a movable element extending portion adjacent to a movable element indented portion, in the manufactured state the movable element extending portion being located opposite a first further element indented portion at a first distance and the movable element indented portion being located opposite a first further element extending portion at a second distance, and wherein the tube is configured to allow relative sideways movement from the manufactured state to the reduced-play state between the movable element and the first further element, wherein in the reduced-play state in which said movable element extending portion is, at least partly, opposite said first further element extending portion such that said movable element extending portion and said first further element extending portion can sideways move relative to one another and slide along one another as caused by operating the cylindrical element until a predetermined maximum operating limit.

2. The cylindrical instrument according to claim 1, wherein the movable element and the first further element are opposite portions of a hinge and the operating of the cylindrical instrument causes a deflection between the opposite portions of the hinge, wherein the predetermined maximum operating limit is a maximum deflection angle between the opposite portions of the hinge.

3. The cylindrical instrument according to claim 2, wherein the maximum deflection angle has a value in a range of at least one of −2 to −45 degrees and +2 to +45 degrees.

4. The cylindrical instrument according to claim 2, wherein the movable element is a convex portion rotatably arranged inside the first further element which is a concave portion.

5. The cylindrical instrument according to claim 4, wherein the convex portion of the movable element has a center point, the movable element extending portion has a height and a curved side facing the first further element and is located on a circle with a radius about the center point, the first further element extending portion has a curved further side which is located on a further circle with a further radius about the center point and in the manufactured state is at a distance from the movable element, wherein the height and distance are equal within manufacturing tolerances.

6. The cylindrical instrument according to claim 1, wherein the movable element is a first longitudinal element extending in the longitudinal direction of the tube and the operating of the cylindrical instrument causes a mutual longitudinal displacement between the first longitudinal element and the first further element and the predetermined maximum operating limit is a maximum mutual longitudinal displacement.

7. The cylindrical instrument of claim 6, wherein the first longitudinal element is attached to a bendable portion of the tube at a distal end of the tube such as to transfer a longitudinal movement of the longitudinal element to a bending of the bendable portion.

8. The cylindrical instrument according to claim 7, wherein the maximum mutual longitudinal displacement has a value in a range of at least one of −0.5 to −40 mm and +0.5 to +40 mm.

9. The cylindrical instrument according to claim 6, wherein first further element extending portion is resilient in a tangential direction of the tube.

10. The cylindrical element according to claim 6, wherein the movable element extending portion and movable element indented portion are part of a plurality of movable element extending portions and movable element indented portions configured such that the movable longitudinal portion is, in general, tapering towards one of its longitudinal ends.

11. The cylindrical element according to claim 6, wherein the first further element is a second longitudinal element extending in the longitudinal direction of the tube.

12. The cylindrical instrument of claim 11, wherein the second longitudinal element is attached to a bendable portion of the tube at a distal end of the tube such as to transfer a longitudinal movement of the second longitudinal element to a bending of the bendable portion.

13. The cylindrical instrument according to claim 1, wherein the movable element has a transition edge portion between the movable element extending portion and the movable element indented portion, which transition edge portion has an obtuse angle relative to the movable element indented portion.

14. The cylindrical instrument according to claim 1, wherein the first further element has a transition edge portion between the first further element extending portion and the first further element indented portion, which transition edge portion has an obtuse angle relative to the first further element indented portion.

15. The cylindrical instrument according to claim 1, wherein the tube has a wall with a thickness in a range of 0.03-2.0 mm.

16. The cylindrical instrument according to claim 1, wherein the tube has a diameter in a range of 0.5-20 mm.

17. A method of manufacturing a cylindrical instrument cutting one or more slots in a first tube such as to produce a first element and a second element in the first tube such that they are movable sideways relative to one another and the first element and second element show play relative to one another; then either performing action
(a) moving at least a portion of the first element and at least a portion of the second element relatively towards one another such that the first element and the second element show reduced play at the location where they have been moved towards one another, and fixing either the portion of the first element or the portion of the second element-such as to keep the status of reduced play while still allowing said sideways relative movement between the first element and the second element during operation of the cylindrical element, or
(b) moving a third element to a predetermined location relative to the first element and to the second element such that the third element remains at the predetermined location and causes reduced play between the first element and the second element.

18. The method according to claim 17, wherein the first element is a first longitudinal element extending in a longitudinal direction of the first tube, the second element is a second longitudinal element extending in the longitudinal direction of the first tube, the first longitudinal element and second longitudinal element being longitudinally movable relative to one another, the method comprising inserting a second tube inside or outside the first tube, the second tube comprising a third element and a fourth element, the third and fourth elements being longitudinally movable relative to one another, implementing action (a) by at least one of attaching the first longitudinal element to the third element by, for instance, at least one of welding, laser welding, brazing, bonding, gluing, bending a folding tab in the first longitudinal element into a recess in the third element, or bending a folding tab in the third element into a recess in the first longitudinal element, or attaching the second longitudinal element to the fourth element by, for instance, at least one of welding, laser welding, brazing, bonding, gluing, bending a folding tab in the first longitudinal element into a recess in the fourth element, or bending a folding tab in the fourth element into a recess in the second longitudinal element.

19. The method according to claim 15, wherein the first tube has a first hinge, the first element is a first convex portion of the first hinge, the second element is a first concave portion in the first hinge, the first convex portion being rotatable in the first concave portion, the method comprising inserting a second tube inside or outside the first tube, the second tube comprising a second hinge, the third element being a second convex portion of the second hinge, the fourth element being a second concave portion of the second hinge, the second convex portion being rotatable in the second concave portion, such that the first convex portion is located adjacent to the second convex portion and the first concave portion is located adjacent to the second concave portion, implementing action (a) by attaching the first convex portion to the second convex portion by, for instance, at least one of welding, laser welding, brazing, bonding, gluing, bending a folding tab in the first convex portion into a recess in the second convex portion, or bending a folding tab in the second convex portion-into a recess in the first convex portion.

20. The method according to claim 15, wherein action (b) is performed and the third element is also produced by the cutting of one or more slots in the first tube.

21. The method according to claim 18, wherein the third element is a spacer and action (b) is implemented by shifting the spacer in a longitudinal direction of the first tube such that the spacer gets fixed in the longitudinal direction by means of a flexible portion in the first element and is pushing against the second element in a tangential direction of the tube.

22. The method according to claim 15, wherein the first tube has a hinge, the first element is a convex portion of the hinge, the second element is a concave portion in the hinge, the convex portion being rotatable in the first concave portion, the method comprising implementing action (a) by moving the convex moving portion and concave portion relative to one another such that they form a circular snap-fit connection.

23. The method according to claim 15, wherein the first tube has a hinge, the first element is a convex portion of the hinge, the second element is a concave portion in the hinge, the convex portion being rotatable in the first concave portion, the method comprising implementing action (a) by arranging a second tube inside or outside the first tube, the second tube being provided with longitudinal elements, moving the convex moving portion and concave portion relative to one another such that they show reduced mutual play and providing the longitudinal elements with a pretension.

24.-34. (canceled)

35. The cylindrical instrument according to claim 1, wherein at least one of the movable element extending portion and the first further element extending portion has a smooth surface.

36. The cylindrical instrument according to claim 35, wherein the movable element is a first longitudinal element extending in the longitudinal direction of the tube and the operating of the cylindrical instrument causes a mutual longitudinal displacement between the first longitudinal element and the first further element and the predetermined maximum operating limit is a maximum mutual longitudinal displacement.
